(19) United States
(12) Patent Application Publication
(10) Pub. No.: US 2024/0141411 A1
(43) Pub. Date: May 2, 2024

(54) MULTIPLEX RNA TARGETING

(71) Applicant: The Jackson Laboratory, Bar Harbor, ME (US)

(72) Inventors: Albert Cheng, Bar Harbor, ME (US); Zukai Liu, Bar Harbor, ME (US)

(73) Assignee: The Jackson Laboratory, Bar Harbor, ME (US)

(21) Appl. No.: 18/280,198

(22) PCT Filed: Mar. 3, 2022
(86) PCT No.: PCT/US2022/018754

§ 371 Date: Sep. 1, 2023

Related U.S. Application Data
(60) Provisional application No. 63/157,088, filed on Mar. 5, 2021.

(51) Int. Cl. C12Q 1/6816; C12N 9/22; C12N 15/115

(52) U.S. Cl. CPC C12Q 1/6816; C12N 9/22; C12N 15/115; C12N 2310/16; C12N 2310/20; C12Y 301/00

(57) ABSTRACT

Provided herein, in some aspects, is a multiplex RNA targeting system that enables live cell imaging and/or modification of multiple RNA targets. Specifically, the disclosure provides a method of live cell imaging of ribonucleic acid (RNA), or targeting RNA in a live cell, comprising: (a) delivering to a cell an RNA-editing complex that comprises a catalytically inactive Cas13 (dCas13) nuclease, a Cas 13 guide RNA (gRNA) comprising an RNA aptamer sequence, and a detectable molecule linked to an RNA-binding domain (RBD), or an RNA effector molecule linked to an RBD sequence that specifically binds to the RNA aptamer sequence; and (b) imaging the detectable molecule or RNA aptamer and RBD binding.

### RELATED APPLICATIONS

This application claims the benefit under 35 U.S.C. § 119(e) of U.S. provisional application No. 63/157,088, filed Mar. 5, 2021, which is incorporated by reference herein in its entirety.

### GOVERNMENT LICENSE RIGHTS

This invention was made with government support under R01-HG009900-01 awarded by National Institutes of Health. The government has certain rights in the invention.

### SEQUENCE LISTING

The instant application contains a Sequence Listing that has been submitted electronically in ASCII format and is hereby incorporated by reference herein in its entirety. Said ASCII copy, created Mar. 3, 2022, is named J022770104WO00-SEQ-EMB.txt and is 64,808 bytes in size.

### BACKGROUND

Post-transcriptional regulation controls gene expression at the RNA level, and its dysfunction is involved in many diseases. It regulates the maturation, chemical modification, stability, localization, and translation of RNAs by a variety of RNA binding proteins. Once transcribed, pre-mRNAs are spliced to remove introns and concatenate exons into one transcript, and a 5′ cap and 3′ poly-A tail are added to produce mature mRNA. The mature mRNAs are then transported from the nucleus to the cytoplasm for translation to produce functional proteins and then degraded as needed. RNA processing steps coordinate together to tightly regulate gene expression, and failure of any step might result in severe disease.

### SUMMARY

Provided herein, in some aspects, is a toolbox that enables multiplex RNA imaging and/or processing. This toolbox leverages the versatility of RNA aptamers and the precision of an engineered RNA-targeting Clustered Regularly Interspaced Palindromic Repeats (CRISPR/Cas) (CRISPR/Cas) system to collectively provide, for example, a sophisticated live cell imaging platform.

The data provided herein demonstrate that with this technology, the guide RNA (gRNA) of an engineered Cas13 variant enzyme can be tagged with different RNA aptamers designed to recruit distinct proteins and/or peptides (e.g., RNA effector molecules) fused with aptamer-binding RNA binding domains (RBDs) (e.g., PUF/MCP/PCP) to execute different RNA binding and/or processing functions (FIG. 1B). By pairing the RNA aptamers on target-specific gRNAs with a cognate RBD-fused proteins (e.g., detectable and/or functional), the methods herein can be used to achieve multi-color imaging of multiple RNAs in the same cell with a single RNA-guided enzyme, and in some embodiments, modulate different RNA processes. In addition, different RNA aptamers can be added to the same gRNAs to coordinate multiple imaging and/or processing steps on a given target or assemble multi-protein complexes. As shown herein, the multiplex RNA system was used to overcome the barrier of non-repetitive RNA sequence labeling by targeting the intron of a gene with multiple copies of the Pumilio binding site motif to image its nascent transcripts. Surprisingly, the mammalian cells co-transfected with the tools of this multiplex RNA system showed bright fluorescent foci in the nuclei, corresponding to the nascent transcripts at the particular gene locus (FIG. 6).

Thus, in some aspects, the present disclosure provides a method of live cell RNA imaging comprising: (a) delivering to a cell an RNA-editing complex that comprising a catalytically inactive Cas13 (dCas13) nuclease, a Cas13 gRNA comprising an RNA aptamer sequence, and a detectable molecule linked to an RBD sequence that specifically binds to the RNA aptamer sequence; and (b) imaging the detectable molecule.

In some embodiments, a dCas13 nuclease is pre-crRNA processing deficient. In some embodiments, a dCas13 nuclease is a dCas13b nuclease. In some embodiments, a dCas13 nuclease is a Prevotella dCas13 nuclease. In some embodiments, a Prevotella dCas13b nuclease is a Prevotella sp. P5-125 dCas13 nuclease (PspdCas13).

In some embodiments, a dCas13 nuclease comprises a mutation at one or more position(s) corresponding to amino acid positions 367-370 of the amino acid sequence of SEQ ID NO: 1. In some embodiments a mutation at one or more position(s) corresponding to amino acid positions 367-370 of SEQ ID NO: 1 is a mutation to a nonpolar neutral amino acid. In some embodiments, a nonpolar neutral amino acid is alanine.

In some embodiments, an RNA aptamer is selected from a Pumilio aptamer sequence, an MS2 aptamer sequence, and a PP7 aptamer sequence. In some embodiments, an RNA aptamer is a Pumilio aptamer sequence and an RBD sequence is a Pumilio binding domain sequence. In some embodiments, an RNA aptamer sequence is an MS2 aptamer sequence and an RBD sequence is an MS2 coat protein (MCP) sequence. In some embodiments, an RNA aptamer sequence is a PP7 aptamer sequence and an RBD sequence is a PP7 coat protein (PCP) sequence.

In some embodiments, a Cas13 gRNA binds to a nonrepetitive RNA sequence.

In some aspects, the present disclosure provides a method of targeting ribonucleic acid (RNA) in a live cell, comprising: (a) delivering to a live cell an RNA-editing complex that comprises a dCas13 nuclease, a Cas13 gRNA comprising an RNA aptamer sequence, and an RNA effector molecule linked to an RNA-binding domain (RBD) sequence that specifically binds to the RNA aptamer sequence, optionally wherein the RNA effector molecule is selected from an RNA splicing factor, an RNA methylation or demethylation protein, an RNA degradation molecule, and an RNA processing molecule; and (b) imaging the detectable molecule.

In other aspects, the present disclosure provides a kit, comprising: a Cas13 gRNA linked to an RNA aptamer sequence; and an RNA effector molecule, optionally a detectable molecule, linked to an RBD sequence that specifically binds to the RNA aptamer sequence.

In some embodiments, the kit further comprises a dCas13 nuclease.

Other aspects provide a multiplex live cell imaging method, comprising transfecting a live cell with: a first Cas13 RNA linked to a first RNA aptamer sequence and a first detectable molecule linked to a first RBD sequence that specifically binds to the first RNA aptamer sequence; and a second Cas13 gRNA linked to a second RNA aptamer sequence and an RNA effector molecule, optionally a second detectable molecule, linked to a second RBD sequence that specifically binds to the second RNA aptamer sequence.

In some embodiments, the method further comprises transfecting the cell with a dCas13 nuclease.

In some embodiments, the cell comprises a first RNA of interest and a second RNA of interest, the first Cas13 gRNA specifically binds to the first RNA of interest, and the second Cas13 gRNA specifically binds to the first second of interest.

In some embodiments, the method further comprises incubating the cell to target, and optionally modify, the first RNA of interest and the second RNA of interest.

Also provided herein in some aspects is a composition comprising: a Cas13 gRNA comprising a Pumilio binding sequence (PBS), and a detectable molecule linked to a Pumilio PBS binding domain (PUF domain).

Also provided herein in some aspects is a composition comprising: a first Cas13 gRNA linked to a first PBS sequence and a first RNA effector molecule, optionally a detectable molecule, linked to a first PUF domain sequence that specifically binds to the first PBS sequence; and a second Cas13 gRNA linked to a second PBS sequence and a second RNA effector molecule, optionally a detectable molecule, linked to a second PUF domain sequence that specifically binds to the second PBS sequence.

In some embodiments, the composition further comprises a dCas13 nuclease.

### BRIEF DESCRIPTION OF THE DRAWINGS

FIGS. 1A-1C. Multiplexed RNA editing leveraging scaffold RNA. (FIG. 1A) Conventional CRISPR/Cas13 mediated RNA editing. gRNA consists of two parts, the spacer binds target RNAs and the directed repeat (DR) binds Cas13 protein. Different effectors can be fused with dCas13 to execute different functions as indicated. (FIG. 1B) In the multiplex RNA targeting system, one or more copies of RNA aptamers are added at the 3′ of gRNA to recruit specific RNA binding domains (RBDs) fused to effector proteins. Example aptamer-RBD pairs are listed on the right. (FIG. 1C) Alignment of PbuCas13b and PspCas13b. The amino acids in rectangles i and iii are mutated to alanine to generate the nuclease-dead dCas13 and the amino acids in rectangle ii are mutated to alanine to disable the crRNA processing activity. The dPspCas13b(AAAA) mutant contains alanine substitutions for all the residues contained in rectangles.

FIGS. 2A-2E. Multiplex RNA targeting system mediated splicing modulation. (FIG. 2A) A diagram of pCI-SMN2 splicing reporter and qPCR primer design. The reporter contains three exons (E6, E7, E8) and two introns of the SMN2 gene. Three gRNAs were designed to target the intron between E7 and E8, indicated by rectangles labeled “gRNA”. The E7 is spliced into the mature transcript of the inclusion isoform and skipped in the exclusion isoform. For qPCR, both transcripts share the same forward primer (SEQ ID NO: 9) but have distinct reverse primers to overlap with the junction of E7 and E8 (SEQ ID NO: 10) or the junction of E6 and E8 (SEQ ID NO: 11), for exclusion and inclusion isoforms respectively. (FIG. 2B) Upper panel shows the schematic of RAS1. One (SEQ ID NO: 44), two (SEQ ID NO: 45) or four copies (SEQ ID NO: 46) of MS2 are added into the gRNA and RBFOX1 is fused with MCP. If not otherwise stated, dCas13 used in this study is dPspCas13b(AAAA). The lower panel shows the inclusion/exclusion (Inc/Exc) ratio assayed by RT-qPCR. C stands for the non-targeting control gRNA and the DN stands for the mixture of three on-target gRNAs. (FIG. 2C) Upper panel shows the schematic of RAS2. Five (SEQ ID NO: 41) or 15 copies (SEQ ID NO: 43) of PBSc (UUGAUGUA) are added into the gRNA and RBFOX1 is fused with PUFc. The lower panel shows the inclusion/exclusion (Inc/Exc) ratio assayed by RT-qPCR. (FIGS. 2D-2E) Comparison of dPspCas13b(AAAA) and dPspCas13b in both RAS1 and RAS2. All fold changes were calculated by normalizing the first column in each plot to 1.

FIGS. 3A-3C. Optimization of PBSc tagged gRNA. (FIGS. 3A-3B) The secondary structure of 3 PBSc with one stem loop and 5 PBSc with two stem loops predicted by online tool called RNAfold (Mathews et al., Proc. Nat. Acad. Sci. USA, 2004) (http://rna.tbi.univie.ac.at/cgi-bin/RNAWebSuite/RNAfold.cgi). (FIG. 3C) The alternative splicing efficacy by modifying the gRNAs in RAS2. C stands for the non-targeting control and DN stands for on-target gRNA. The numbers indicate how many PBSc in the RNA scaffold while the ‘loop’ marks the scaffolds with synthetic stem loops. Fold change was calculated by normalizing the first column (C-0) to 1.

FIGS. 4A-4B. Recognition between gRNA-aptamers and their RBD-effector is specific and orthogonal. (FIG. 4A) All groups were transfected with dpspCas13b and MS2 tagged gRNAs. The groups on the left were co-transfected with MCP-RBFOX1 while the groups on the right were co-transfected with PUFc-RBFOX1. (FIG. 4B) All groups were transfected with dPspCas13b and PBSc tagged gRNAs. The groups on the left were co-transfected with MCP-RBFOX1 while the groups on the right were co-transfected with PUFc-RBFOX1. Inc/Exc was assayed by RT-qPCR. All fold changes were calculated by normalizing the first column in each plot to 1.

FIGS. 5A-5C. Site-specific RNA m6A modification. (FIG. 5A) Design of gRNA targeting to A1216 in ACTB mRNA. All gRNAs are 22 nucleotides except T, which is 30 nucleotides. (FIGS. 5B-5C) Upper panel, the schematic directed m6A modification components mediated by PUFc-M3 and PUFa-M3 respectively. The gRNAs were tagged with 15×PBSc or 5×PBSa respectively. Middle panel, the relative mRNA level of ACTB with different gRNAs, normalized by the non-target control. Bottom panel, relative m6A level assayed by SELECT-PCR. 1 ug total RNA was used for each sample.

FIG. 6. RNA live-cell imaging. HEK293T cells were transfected with dPspCas13b, Clover-NLS-PUFc and gRNAs targeting intron of LMNA. Dotted lines mark the boundaries of nuclei and arrows point to the fluorescent foci labeling the nascent LMNA transcript at the LMNA loci.

### DETAILED DESCRIPTION

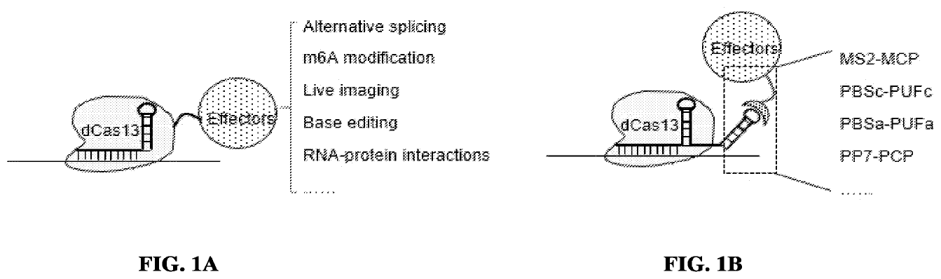

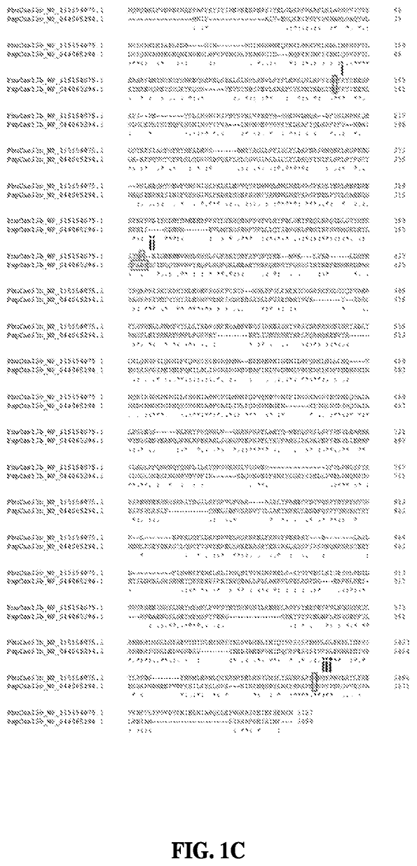

Provided herein, in some aspects, are methods and compositions for multiplexed RNA imaging in live cells using a (CRISPR/Cas) RNA targeting system. As shown in FIG. 1B, in one embodiment, this tripartite system comprises (a) a catalytically inactive, pre-crRNA processing deficient RNA-guided nuclease, such as a dCas13 nuclease (e.g., dCas13b), (b) a guide RNA (gRNA) comprising an RNA aptamer sequence, and (c) a protein (e.g., an RNA imaging or effector molecule) comprising an RNA binding domain (RBD) sequence that specifically binds to the RNA aptamer sequence. A complex is formed at the target site of interest upon binding of the RBD sequence (and thus the effector molecule) to the RNA aptamer sequence of the gRNA.

The technology provided herein fills a gap in live cell RNA imaging. While fluorescence in situ hybridization techniques have been widely used to study RNA, the requirement for cell fixation has prohibited dynamic RNA imaging. dCas9-gRNA systems have also been utilized to image non-repetitive genomic loci, but these systems are difficult to adapt for live cell imaging because of the need to deliver dozens of gRNAs into cells and the accompanying increase in off-target imaging. Additionally, while several RNA aptamers and their RBDs have been developed in the last several decades, including the MS2 aptamer and MS2 coat protein (MCP) system and the PP7 aptamer and PP7 coat protein (PCP) system (e.g., Keryer-Bibens et al., Biol. Cell., 2008), their target sequence diversity has remained limited (e.g., Choudhury et al., Nat. Commun. 2012; Wang et al., Nat. Methods, 2013). Also, these RNA aptamer sequences must generally be inserted onto an RNA of interest to generate chimeric transcripts for targeting, which makes targeting of endogenous RNAs challenging, particularly for live cell imaging applications. The multiplex RNA targeting system provided herein overcomes these challenges by utilizing the large sequence diversity present in the Pumilio aptamer system, for example, and incorporating RNA aptamer sequences onto an RNA-guided RNA-editing (e.g., Cas13) scaffolding gRNA. Multiple RNA aptamer sequences may be incorporated onto a gRNA, allowing imaging of numerous RNA molecules in a live cell.

### Multiplex RNA Targeting Systems

Provided herein is a multiplex RNA targeting system that leverages the versatility of RNA aptamers and the precision of an engineered RNA-targeting CRISPR/Cas (e.g., Cas13) system. This system may be used for any RNA targeting function. Non-limiting examples of RNA targeting functions include: imaging, splicing, methylation, demethylation, editing, and processing.

### CRISPR/Cas RNA Targeting Systems

In some aspects of the present disclosure, a CRISPR/Cas RNA targeting system herein contains a Cas nuclease enzyme with RNAse activity, a scaffold guide RNA (gRNA) that guides the Cas nuclease enzyme to a target RNA sequence, the target RNA sequence that the Cas nuclease enzyme binds, and an RNA effector molecule. The terms “Cas nuclease,” “Cas enzyme,” and “Cas protein” are used interchangeably herein. CRISPR/Cas nucleases are well-known in the art (e.g., Harrington, L. B. et al., Science, 2018) and exist in a variety of bacterial species where they recognize and cut specific nucleic acid (e.g., RNA or DNA) sequences. CRISPR/Cas nucleases are grouped into two classes. Class I systems use a complex of multiple CRISPR/Cas proteins to bind and degrade nucleic acids, whereas Class II systems use a single, large protein for the same purpose. In some embodiments, a Cas nuclease of the present disclosure is a Class II nuclease that binds and degrades nucleic acid (e.g., RNA).

### Nucleases

A Cas nuclease may be any naturally occurring or engineered Cas nuclease with RNAse activity or that can otherwise form a complex with a gRNA to bind to an RNA target of interest. Non-limiting examples of Cas nucleases include: Cas1, Cas2, Cas3, Cas4, Cas5, Cas6, Cas7, Cas8, Cas9, Cas10, Cas 11, Cas12, and Cas13. Cas13, for example, naturally has RNase activity.

CRISPR/Cas nucleases from different bacterial species have different properties (e.g., specificity, activity, binding affinity). Non-limiting examples of bacteria from which Cas nuclease may be derived include: Prevotella (e.g., Prevotella sp. P5-125, Prevotella buccae), Staphylococcus (e.g., Staphylococcus aureus, Staphylococcus epidermidis), Streptococcus (e.g., Streptococcus pyogenes, Streptococcus thermophilus), Neissseria (e.g., Neisseria meningitidis, Neisseria gonorrhoeae), Porphyromonas (e.g., Porphyromonas gulae, Porphyromonas gingivalis) Riemerella (e.g., Riemerella anatipestifer, Riemerella columbipharyngis), Leptotrichia (e.g., Leptotrichia wadei, Leptotrichia buccalis, Leptotrichia shahii), Ruminococcus (e.g., Ruminococcus flavefaciens, Ruminococcus productus) Bergeyella (e.g., Bergeyella zoohelcum, Bergeyella cardium), and Listeria (e.g., Listeria seeligeri, Listeria monocytogenes).

In some embodiments, a Cas nuclease is a Cas13 nuclease. Cas13 nuclease lacks a DNase domain compared to other Cas nucleases and instead contains two higher eukaryote and prokaryote nucleotide (HEPN) RNAse domains. Cas13 nuclease binds to a guide RNA known as CRISPR-RNA (crRNA) and then undergoes a conformational change that brings the two HEPN domains together to form a single catalytic site with RNAse activity (e.g., Slaymaker, et al., Cell Reports, 2019; Liu, et al., Cell, 2017). This conformational activation of RNAse activity is advantageous for Cas13 because after it binds a target RNA sequence, it can also destroy nearby RNA nucleotides that are not part of the target nucleotide sequence (e.g., Pawluck, Cell, 2020). In addition to RNAse catalytic activity, Cas13 nucleases also possess catalytic crRNA maturation activity in which precursor crRNAs are processed into active crRNAs. crRNA maturation catalytic activity is discussed in greater detail below.

A Cas13 nuclease used herein is not limited to any particular bacterial species. In some embodiments, a Cas13 nuclease is a Prevotella Cas13 nuclease. A Prevotella Cas13 nuclease protein may be from any Prevotella species. Non-limiting examples of Prevotella species include Prevotella (P.) sp. P5-125, P. albensis, P. amnii, P. bergensis, P. bivia, P. brevis, P. bryantii, P. buccae, P. buccalis, P. copri, P. dentalis, P. denticola, P. disiens, P. histicola, P. intermedia, P. maculosa, P. marshii, P. melaninogenica, P. micans, P. multiformis, P. nigrescens, P. oralis, P. oris, P. oulorum, P. pallens, P. salivae, P. stercorea, P. tannerae, P. timonensis, and P. veroralis. In some embodiments, a Prevotella Cas13 nuclease is a Prevotella sp. P5-125 Cas13 nuclease (PspCas13).

Further, a Cas13 nuclease used herein is not limited by any particular subtype. Non-limiting examples of Cas13 nuclease subtypes include Cas13a (C2c2), Cas13b (C2c6), Cas13c (C2c7), and Cas13d. These Cas13 nuclease subtypes are distinguished based on their size, the composition of their protein domains, and the configuration of crRNAs that they bind. In some embodiments, a Cas13 nuclease is a Cas13b nuclease.

In some embodiments, a Cas nuclease is catalytically inactive (e.g., dCas). A catalytically inactive Cas nuclease herein includes any of the recombinant or naturally occurring forms of the Cas nuclease or variants or homologs thereof that are modified to be catalytically inactive (e.g., within at least 50%, 80%, 90%, 95%, 96%, 97%, 98%, 99%, or 100% activity compared to Cas). In some aspects, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring Cas nuclease. A Cas nuclease may be made catalytically inactive by point mutations, combinations of mutations, or elimination or substitution of one or more catalytic (e.g., RNAse) domains.

In some embodiments, a catalytically inactive Cas nuclease is a catalytically inactive Cas13 nuclease. These catalytically inactive ‘dead’ Cas13 (dCas13) proteins can be fused with other effector proteins for manipulating different RNA processing steps instead of target RNA cleavage (FIG. 1A). Mutations of catalytic residues in the HEPN domains deactivates Cas13 nuclease activity, while retaining RNA binding activity. These mutations in the HEPN domains may be single point mutations, combinations of point mutations, insertions, or deletions such that the Cas13 nuclease retains RNA-binding activity. These mutations may be conservative (e.g., positively charged amino acid for positively charged amino acid) or non-conservative (e.g., neutral, nonpolar amino acid for positively charged amino acid).

In some embodiments, a dCas13 nuclease is a dCas13a nuclease, a dCas13b nuclease, a dCas13c nuclease, or a dCas13d nuclease. In some embodiments, a catalytically inactive dCas13 nuclease is a dCas13b nuclease with the amino acid sequence in SEQ ID NO: 1. In some embodiments, a dCas13b nuclease has a modified version of the amino acid sequence in SEQ ID NO: 1.

In some embodiments, a catalytically inactive Cas13 nuclease is a Cas13b nuclease (dCas13b). In some embodiments, a dCas13 nuclease is a Prevotella sp. P5-125 dCas13b nuclease (PspdCas13).

In some embodiments, a Cas13 nuclease is catalytically inactive because Cas13 nuclease proteins possess non-specific RNAse activity as described above.

Active CRISPR-RNAs (crRNAs) are produced from a CRISPR precursor transcript (pre-crRNA). In a cell, arrays of pre-crRNAs may be transcribed in a single nucleic acid molecule, and the resulting pre-crRNA is processed (matured) by Cas nucleases and other RNA endonuclease proteins into a set of crRNA molecules. A set of crRNA molecules may include 1-50, 5-40, 5-30, 5-20, 5-10, 10-50, 10-40, 10-30, 10-20, 20-50, 20-40, 20-30, 30-50, 30-40, 40-50 or more crRNA molecules. Mature crRNA molecules contain of a single spacer sequence and a repeat sequence. Mature crRNA molecules are bound by Cas nucleases.

An RNA endonuclease protein that processes pre-crRNA into crRNA may be any RNA endonuclease protein, including certain Cas nucleases. Non-limiting examples of RNA endonuclease proteins include: Cas13, Cse (CasE), Cas6, Cys4, Cas5d, RNAse I, RNAseII, and RNAse III.

In some embodiments, an RNA endonuclease protein that processes pre-crRNA into crRNA is a Cas13 nuclease. Cas13a, Cas13c, and Cas13d nucleases process pre-crRNA into crRNAs with a direct repeat (DR) region and a spacer region (5′ to 3′). Cas13b nuclease processes pre-crRNA into crRNAs with a spacer region and a direct repeat region (5′ to 3′). However, pre-crRNA processing is not required when a Cas nuclease is a Cas13 nuclease, and pre-crRNA is sufficient for Cas13 nuclease binding a target RNA sequence (e.g., East-Seletsky, et al., Molecular Cell, 2017).

In some embodiments, a Cas nuclease herein is pre-crRNA processing deficient. A pre-crRNA processing deficient Cas nuclease herein includes any of the recombinant or naturally occurring forms of the Cas nuclease or variants or homologs thereof that are modified to be pre-crRNA processing deficient (e.g., within at least 50%, 80%, 90%, 95%, 96%, 97%, 98%, 99%, or 100% activity compared to naturally occurring Cas nuclease). In some aspects, the variants or homologs have at least 90%, 95%, 96%, 97%, 98%, 99%, or 100% amino acid sequence identity across the whole sequence or a portion of the sequence (e.g., a 50, 100, 150, or 200 continuous amino acid portion) compared to a naturally occurring Cas nuclease. A Cas nuclease may be made pre-crRNA processing by point mutations, combinations of mutations, or elimination or substitution of one or more pre-crRNA processing (e.g., lid) domains.

Pre-crRNA processing deficiency in a Cas nuclease herein is distinct from catalytic inactivity and may be in addition to or independent of catalytic inactivity. In some embodiments, a Cas nuclease (e.g., Cas13) is catalytically inactive (dCas) and pre-crRNA processing deficient.

A Cas nuclease that is pre-crRNA processing deficient is preferable when pre-crRNA does not need to be processed into crRNAs for Cas nuclease activity to occur.

In some embodiments, a pre-crRNA processing deficient Cas13 nuclease is a Cas13b nuclease. In Cas13b nuclease, amino acids responsible for pre-crRNA processing activity are in the lid domain (e.g., Slaymaker, et al., Cell Reports, 2019). Thus, any residues or any combination of residues in a lid domain or suspected lid domain of a Cas13 nuclease may be mutated or deleted to render the Cas13 nuclease pre-crRNA processing deficient. Sequences of Cas13 nuclease (e.g., Cas13b nuclease) may be aligned to identify residues in the lid domain that are required for pre-crRNA processing. Any combination of these residues may be mutated to render the Cas13 nuclease pre-crRNA processing deficient.

In some embodiments, a pre-crRNA processing deficient Cas13 nuclease is a Prevotella (P.) sp. P5-125 Cas13b nuclease. The lid domain in P. sp. P5-125 contains amino acids 367-370 (KADK) that are critical to pre-crRNA processing. In some embodiments, one, some, or all of amino acids 367-370 are mutated to render P. sp. P5-125 pre-crRNA processing deficient. Thus, in some embodiments, a pre-crRNA processing deficient enzyme has a mutation at one or more position(s) corresponding to amino acid positions 367-370 (KADK) of SEQ ID NO: 1. In some embodiments, a pre-crRNA processing deficient enzyme has the amino acid sequence of SEQ ID NO: 2.

In some embodiments, one position, two positions, three positions, or four positions corresponding to amino acid positions 367-370 of SEQ ID NO: 1 are mutated in a pre-crRNA processing deficient Cas nuclease (e.g., Cas13b) in the present disclosure. In some embodiments, an amino acid corresponding to amino acid position 367 of SEQ ID NO: 1 is mutated. In some embodiments, an amino acid corresponding to amino acid position 368 of SEQ ID NO: 1 is mutated. In some embodiments, an amino acid corresponding to amino acid position 369 of SEQ ID NO: 1 is mutated. In some embodiments, an amino acid corresponding to amino acid position 370 of SEQ ID NO: 1 is mutated. In some embodiments, amino acids corresponding to amino acid positions 367 and 368 of SEQ ID NO: 1 are mutated. In some embodiments, amino acids corresponding to amino acid positions 367 and 369 of SEQ ID NO: 1 are mutated. In some embodiments, amino acids corresponding to amino acid positions 367 and 370 of SEQ ID NO: 1 are mutated. In some embodiments, amino acids corresponding to amino acid positions 368 and 369 of SEQ ID NO: 1 are mutated. In some embodiments, amino acids corresponding to amino acid positions 368 and 370 of SEQ ID NO: 1 are mutated. In some embodiments, amino acids corresponding to amino acid positions 367, 368 and 369 of SEQ ID NO: 1 are mutated. In some embodiments, amino acids corresponding to amino acid positions 367, 369 and 370 of SEQ ID NO: 1 are mutated. In some embodiments, amino acids corresponding to amino acid positions 368, 369 and 370 of SEQ ID NO: 1 are mutated. In some embodiments, amino acids corresponding to amino acid positions 367, 368, 369 and 370 of SEQ ID NO: 1 are mutated.

In some embodiments, one or more position(s) corresponding to amino acid positions 367-370 of SEQ ID NO: 1 (KADK) are mutated to a nonpolar neutral amino acid. Non-limiting examples of nonpolar neutral amino acids are alanine (A), valine (V), leucine (L), isoleucine (I), proline (P), phenylalanine (F), methionine (M), tryptophan (W), glycine (G), and cysteine (C). In some embodiments, one or more position(s) corresponding to amino acid positions 367-370 of SEQ ID NO: 1 are mutated to alanine. In some embodiments a pre-crRNA processing deficient enzyme has the amino acid sequence of SEQ ID NO: 2. In some embodiments, one or more position(s) corresponding to amino acid positions 367-370 of SEQ ID NO: 1 are mutated to a combination of nonpolar neutral amino acids.

In some embodiments, an amino acid corresponding to amino acid position 367 of SEQ ID NO: 1 is mutated to a nonpolar neutral amino acid (e.g., alanine). In some embodiments, an amino acid corresponding to amino acid position 368 of SEQ ID NO: 1 is mutated to a nonpolar neutral amino acid (e.g., alanine). In some embodiments, an amino acid corresponding to amino acid position 369 of SEQ ID NO: 1 is mutated to a nonpolar neutral amino acid (e.g., alanine). In some embodiments, an amino acid corresponding to amino acid position 370 of SEQ ID NO: 1 is mutated to a nonpolar neutral amino acid (e.g., alanine). In some embodiments, amino acids corresponding to amino acid positions 367 and 368 of SEQ ID NO: 1 are mutated to one or more nonpolar neutral amino acids (e.g., alanine). In some embodiments, amino acids corresponding to amino acid positions 367 and 369 of SEQ ID NO: 1 are mutated to one or more nonpolar neutral amino acids (e.g., alanine). In some embodiments, amino acids corresponding to amino acid positions 367 and 370 of SEQ ID NO: 1 are mutated to one or more nonpolar neutral amino acids (e.g., alanine). In some embodiments, amino acids corresponding to amino acid positions 368 and 369 of SEQ ID NO: 1 are mutated to one or more nonpolar neutral amino acids (e.g., alanine). In some embodiments, amino acids corresponding to amino acid positions 368 and 370 of SEQ ID NO: 1 are mutated to one or more nonpolar neutral amino acids (e.g., alanine). In some embodiments, amino acids corresponding to amino acid positions 367, 368 and 369 of SEQ ID NO: 1 are mutated to one or more nonpolar neutral amino acids (e.g., alanine). In some embodiments, amino acids corresponding to amino acid positions 367, 369 and 370 of SEQ ID NO: 1 are mutated to one or more nonpolar neutral amino acids (e.g., alanine). In some embodiments, amino acids corresponding to amino acid positions 368, 369 and 370 of SEQ ID NO: 1 are mutated to one or more nonpolar neutral amino acids (e.g., alanine). In some embodiments, amino acids corresponding to amino acid positions 367, 368, 369 and 370 of SEQ ID NO: 1 are mutated to one or more nonpolar neutral amino acids (e.g., alanine).

### Guide RNA

CRISPR/Cas nucleases are directed to a target site of interest through complementary base pairing between the target site and a guide RNA (gRNA). The terms “gRNA” and “crRNA” are used interchangeably herein. A gRNA herein comprises (1) at least one user-defined spacer sequence (also referred to as an RNA-targeting sequence) that hybridizes to (binds to) a target RNA sequence (e.g., non-coding sequence, coding sequence) and (2) a scaffold sequence (e.g, a direct repeat sequence) that binds to the CRISPR/Cas nuclease to guide the CRISPR/Cas nuclease to the target RNA sequence. As is understood by the person of ordinary skill in the art, each gRNA is designed to include a spacer sequence complementary to its target RNA sequence. The length of the spacer sequence may vary, for example, it may have a length of 15-50, 15-40, 15-30, 20-50, 20-40, or 20-30 nucleotides. In some embodiments, the length of a spacer sequence is 15, 16, 17, 18, 19, 20, 21, 22, 23, 24, 25, 26, 27, 28, 29, or 30+/−2 nucleotides.

In some embodiments, a CRISPR/Cas system is a CRISPR/Cas13 system, and a gRNA is a Cas13 gRNA. A CRISPR/Cas13 system gRNA comprises a direct repeat (DR) hairpin structure that binds to a Cas13 nuclease and a spacer sequence that binds to a complementary RNA target sequence. A direct repeat hairpin structure may be upstream (towards the 5′ end) of the spacer sequence (e.g., in a Cas13a, Cas13c, or Cas13d nuclease system) or downstream (towards the 3′ end) of the spacer sequence (e.g., in a Cas13b nuclease system) in a gRNA of the present disclosure.

### RNA Aptamers and RBD Sequences

Guide RNAs provided in the present disclosure comprise RNA aptamers. An RNA aptamer is an RNA sequence (e.g., a single-stranded RNA sequence, a double-stranded RNA sequence, a hybrid single-stranded RNA sequence, or a partially double-stranded RNA sequence) that can be recognized and bound by particular RNA binding domains (RBDs). In the present disclosure, an RNA aptamer binds to an RBD. RNA aptamers and RBDs are not limited to specific RNA aptamers and RBDs. Non-limiting examples of RNA aptamers are PUF-domain binding (PBS) sequences, MS2 sequences, PP7 sequences, Qβ sequences, A30 sequences, J-18 sequences, CD4 sequences, A10 sequences, and PRR scaffold binding sequences (e.g., Germer, et al., Int. J. Biochem. Mol. Biol., 2013). Non-limiting examples of RBDs are Pumilio-FBF (PUF) domains, MS2 coat protein (MCP) domains, PP7 coat protein (PCP) domains, RNA recognition motifs (RRMs), K-homology domains (KH), RGG (Arg-Gly-Gly) box, zinc finger domains, double stranded RNA-binding domains (dsRBDs), Piwi/Argonaute/Zwille (PAZ) domains, and PRR scaffold domains (see, e.g., Coquille S et al. Nature Communications 20014; 5(5729)).

In some embodiments, an RNA aptamer sequence is a PUF-domain binding sequence (PBS) and an RBD sequence is a PUF domain. PUF domains and PBSs are known in the art (see e.g., International Publication No. WO2016148994A and Cheng A. et al. Cell Research 2016; 26: 254-257, each of which is incorporated herein by reference). Briefly, a PBS is bound by a PUF domain. In some embodiments, a PBS is an 8-mer. In such embodiments, there are more than 65,000 possible PBS sequences (given 4 possible RNA nucleotides). In other embodiments, a PBS of the present disclosure has 5, 6, 7, 8, 9, 10, 11, 12, 13, 14, 15, 16 or more RNA nucleotides. PUF domains contain multiple tandem repeats of 35-39 amino acids that recognize specific RNA bases. In some embodiments, a PUF domain of the present disclosure binds 5, 6, 7, 5 8, 9, 10, 11, 12, 13, 14, 15, 16 or more RNA nucleotides in a PBS. In some embodiments, a PUF domain is composed of more than 8 units. For example, PUF9R has 9 units and recognizes 9 RNA bases. See, e.g., Zhao Yet al., Nucleic Acids Research, 2018; 46(9): 4771-4782).

A PBS and a PUF domain may be any PBS and its corresponding PUF domain. In some embodiments, a PBS of the present disclosure has the sequence 5′-UGUAUAUA-3′ and binds the wild-type human Pumilio 1 PUF domain. In some embodiments, the PBS of the present disclosure has the sequence 5′-UGUAUGUA-3′ and binds the PUF domain PUF(3-2). In some embodiments, the PBS of the present disclosure has the sequence 5′-UUGAUAUA-3′ and binds the PUF domain C. In some embodiments, the PBS of the present disclosure has the sequence 5′-UGGAUAUA-3′ and binds the PUF domain PUF(6-2). In some embodiments, the PBS of the present disclosure has the sequence 5′-UUUAUAUA-3′ and binds the PUF domain PUF(7-2). In some embodiments, the PBS of the present disclosure has the sequence 5′-UGUGUGUG-3′ and binds the PUF domain PUF531. In some embodiments, the PBS of the present disclosure has the sequence 5′-UGUAUAUG-3′ and binds the PUF domain PUF(1-1). In some embodiments, the PBS of the present disclosure has the sequence 5′-UUUAUAUA-3′ or 5′-UAUAUAUA-3′ and binds the PUF domain PUF(7-1). In some embodiments, the PBS of the present disclosure has the sequence 5′-UGUAUUUA-3′ and binds the PUF domain PUF(3-1). In some embodiments, the PBS of the present disclosure has the sequence 5′-UUUAUUUA-3′ and binds the PUF domain PUF(7-2/3-1). In some embodiments, the PBS of the present disclosure has the sequence 5′-UUGAUGUA-3′ and binds the PUF domain PUFc. In some embodiments, the PBS of the present disclosure has the sequence 5′-UGUUGUAUA-3′ and binds the PUF domain PUF9R. Any one of the PUF domains described in WO 2016148994 may be used as provided herein. Other PUF domains may be used.

In some embodiments, the RNA aptamer sequence is an MS2 aptamer sequence and the RBD sequence is an MCP sequence. MS2 aptamers and MCP sequences are known in the art (e.g., Bertrand, et al., Molecular Cell, 1998). Briefly, MS2 aptamers sequences are RNA sequences derived from the bacteriophage MS2 and form stem loops that are recognized by the MS2 coat protein (MCP) binding sequences. MCP RBDs preferentially bind RNA stem loops with a bulged purine (e.g., non-paired adenine (A) or uracil (U)) separated by 2 base pairs from a second stem loop. Any MS2 aptamer sequence and its corresponding MCP sequence may be used.

In some embodiments, the RNA aptamer sequence is an PP7 aptamer sequence and the RBD sequence is a PCP sequence. PP7 aptamers and PCP sequences are known in the art (e.g., Lim and Peabody, Nucl. Acids Res., 2002). Briefly, PP7 aptamers are RNA sequences derived the bacteriophage PP7 and form stem loops that are recognized by the PP7 coat protein (PCP) binding sequences. PCP RBDs bind RNA stem loops with a bulged purine (e.g., non-paired A or U) on their 5′ side separated by 4 base pairs from a second RNA stem loop. Any PP7 aptamer sequence and its corresponding PCP sequence may be used.

### RNA Aptamers

In some embodiments, a gRNA of the present disclosure further comprises an RNA aptamer sequence. It will be understood that “an RNA aptamer sequence” refers to one or more RNA aptamer sequences. An RNA aptamer may be linked to or incorporated within a gRNA. “Linked to” in this context refers to an RNA aptamer attached to (joined to) the 5′ end or the 3′ end of the gRNA or inserted internally (between the 5′ end the 3′ end of the gRNA). An RNA aptamer linked to a gRNA may be directly linked with no intervening linker or indirectly linked through an intervening linker to a gRNA. An intervening linker may be any linker including, but not limited to: a nucleotide sequence (e.g., RNA, DNA, RNA/DNA), a polypeptide sequence that is either cleavable (e.g., by an endonuclease) or non-cleavable, or a disulfide linker. Other linkers may also be used.

An RNA aptamer sequence incorporated within a gRNA may be located anywhere within the gRNA such that a Cas nuclease can still bind the gRNA (e.g., at a direct repeat sequence) and the spacer sequence can still bind to its target RNA sequence. In some embodiments, an RNA aptamer sequence may be located upstream of (5′ to), within, or downstream of (3′ to) a repeat sequence that binds to a Cas nuclease. In some embodiments, an RNA aptamer sequence may be located upstream of (5′ to), within, or downstream of (3′ to) a spacer sequence. In some embodiments, an RNA aptamer sequence is located between the direct repeat sequence and the spacer sequence.

A gRNA of the present disclosure may contain any number of RNA aptamers. In some embodiments, a gRNA contains 1-50, 1-40, 1-30, 1-20, 1-10, 1-5, 5-50, 5-40, 5-30, 5-20, 5-10, 10-50, 10-40, 10-30, 10-20, 20-50, 20-40, 20-30, 30-50, 30-40, or 40-50 RNA aptamers. When multiple RNA aptamers are present on the same gRNA, the RNA aptamers may all bind the same RNA-binding domain (RBD), some may bind different RBDs, or they may all bind to different RBDs. The presence of multiple RNA aptamers on a single gRNA allows for multiplexing at a target RNA molecule because each RNA aptamer will be bound by a single RNA binding domain (RBD) sequence.

In embodiments where more than one RNA aptamer is present an on single gRNA, one or more spacer region(s) may separate two adjacent RNA aptamers. The spacer regions may have a length of from about 3 nucleotides to about 100 nucleotides. For example, the spacer can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 10 3 nucleotides (nt) to about 10 nt. For example, the spacer can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the spacer is 4 nt.

### RNA Effector Molecules

CRISPR/Cas RNA systems of the present disclosure comprise an RNA effector molecule. An RNA effector herein refers to a molecule (e.g., a protein or peptide) that can be detected (e.g., imaged) and/or that acts on a target RNA. Non-limiting examples of RNA effector molecule functions include transcriptional regulatory functions (e.g., splicing, expression), post-transcriptional modification functions (e.g., methylation, demethylation), and other RNA processing functions (e.g., targeting (e.g, for degradation)).

In some embodiments, an RNA effector molecule is a detectable molecule. A detectable molecule is a molecule that may be tracked in a cell. Tracking of a detectable molecule may be by any method including, but not limited to: imaging, scanning, and microscopy. In some embodiments, a detectable molecule is imaged in a cell. Imaging may be in a live cell or in a dead cell (e.g., fixed cell) either in vitro or in vivo. Methods of imaging a detectable molecule in a cell include, but are not limited to: fluorescence, radiolabeled emission, heavy atom labeling, and electron microscopy.

In some embodiments, RNA effector molecules are detectable molecules that are imaged by fluorescence. Fluorescence imaging relies on fluorescent proteins and/or fluorescent dyes. An RNA effector molecule may be any fluorescent protein or fluorescent dye. Non-limiting examples of fluorescent proteins include: green fluorescent protein (GFP), red fluorescent protein (RFP), yellow fluorescent protein (YFP), Clover, Sirius, blue fluorescent protein (BFP), SBFP2, Azurite, mAzurite, EBFP2, moxBFP, mKalama1, mTagBFP2, Aquamarine, cyan fluorescent protein (CFP), ECFP, Cerulean, mCerulean3, moxCerulean3, SCFP3A, mTurqouise2, CyPet, AmCyan1, MiCy, iLOV, AcGFP1, sfGFP, moxGFP, mEmerald, EGFP, mEGFP, mAzamiGreen, CfSGFP2, ZsGreen, SGFP2, mClover2, mClover3, mNeonGreen, EYFP, Topaz, mTopaz, mVenus, moxVenus, SYFP2, mGold, mCitrine, yPet, ZsYellow, mPapayay1, mCyRFP1, mKO, mOrange, mOrange2, mKO2, TurboRFP, tdTomato, mScarlet-H, mNectarine, mRuby2, eqFP611, DsRed2, mApple, mScarlet, mScarlet-1, mStrawberry, FusionRed, mRFP1, mCherry, and mCherry2. Non-limiting examples of fluorescent dyes include: AlexaFluor 350, Alexa Fluor 405, Alexa Fluor 488, Alexa Fluor 532, Alexa Fluor 546, Alexa Fluor 555, Alexa Fluor 561, Alexa Fluor 568, Alexa Fluor 594, Alexa Fluor 647, Alexa Fluor 660, Alexa Fluor 680, Alexa Fluor 700, Alexa Fluor 750, Pacific Blue, Coumarin, BODIPY-FL, Pacific Green, Oregon Green, Fluorescein (FITC), Cy3, Pacific Orange, PE-Cyanine 7, PerCP-Cyanine 5.5, Tetramethylrhodamine (TRITC), Texas Red, Cy5, SNAP-tag, CLIP-tag, and HALO-tag.

Any combination of these fluorescent proteins and/or fluorescent dyes may be used in methods, kits, and compositions provided herein. For example, utilizing multiple different fluorescent proteins will allow imaging of multiple RNA effector molecules at a given RNA molecule or at multiple RNA molecules simultaneously in a living cell.

In some aspects of the present disclosure, an RNA effector molecule is an RNA splicing factor. An RNA splicing factor is a protein that transforms precursor messenger RNA (pre-mRNA) into mature mRNA. During splicing, introns (e.g., protein non-coding regions) are removed from pre-mRNA and exons (e.g., protein coding regions) are joined (spliced) together. Alternative splicing occurs when exons are joined together at various sequences or in various configurations (e.g., Clancy, Nature Education, 2008). Non-limiting examples of splicing factors include: RBFOX1, U2 small nuclear RNA auxiliary factor 1 (U2AF35), U2AF2 (U2AF65), splicing factor 1 (SF1), U1 small nuclear ribonucleoprotein (snRNP), U2 snRNP, U4 snRNP, U5 snRNP, U6 snRNP, U11, U12, U4atac, and U6atac. Any combination of these splicing factors or any other splicing factor may be used in methods, kits, and compositions provided herein.

In some aspects of the present disclosure, an RNA effector molecule is an RNA methylation or demethylation protein. RNA methylation is a post-transcriptional modification (e.g., Zhou, et. al., Biomedicine & Pharmacotherapy, 2020). RNA methylation and demethylation influence gene expression, protein translation, and pathological states including cancer, immunity, and response to viral infection. An RNA molecule may be methylated at any site including, but not limited to: the sixth N of adenylate (m6A), at the first N of adenylate (m1A), at the fifth N of cytosine (m5C). An RNA methylation protein may be any protein involved in methylating or demethylating RNA including, but not limited to: METTL3, METTL14, WTAP, VIRMA, ZC3H13, RBM15, RBM15B, HAKAI, METTL16, METTL5, FTO, and ALKBH5. Any combination of these RNA methylation or demethylation proteins or any other RNA methylation proteins or demethylation proteins may be used in methods, kits, and compositions provided herein.

In some aspects of the present disclosure, an RNA effector molecule is an RNA degradation molecule. An RNA degradation molecule is a molecule that mediates the degradation of a target RNA. RNAs are degraded at various times depending on their function, with ribosomal RNAs having a long existence and RNA molecules with defects in processing, folding, or assembly having very short existences (e.g., Dey and Jaffrey, Cell Chemical Biology, 2019). An RNA degradation molecule may be any molecule including, but not limited to: proteins including Rnt1p; chimeras including ribonuclease targeting chimeras (RIBOTACs), (2′-5′)oligoadenylate antisense chimera; and small molecules including Targapremir-210 (TGP-210). Any combination of these RNA degradation molecules or any other RNA degradation molecules may be used in methods, kits, and compositions provided herein.

In some aspects of the present disclosure, an RNA effector molecule is an RNA processing molecule. RNA processing includes mRNA 5′ capping, mRNA 3′ polyadenylation, and/or histone mRNA processing (e.g., Lodish et al., Molecular Cell Biology, 4^{th} edition, 2000). Non-limiting examples of RNA processing molecules include: RNA triphosphatase, guanosyl transferase, guanine-N⁷-methyltransferase, cleavage and polyadenylation specificity factor, cleavage stimulation factor, cleavage factor 1, polyadenylate polymerase, cleavage and polyadenylation specificity factor 73. Any combination of these RNA processing molecules or any other RNA processing molecules may be used in methods, kits, and compositions provided herein.

### Aptamer-Binding RBD Sequences

In some embodiments, an RNA effector molecule of the present disclosure further comprises an aptamer-binding RNA binding domain (RBD) sequence. It will be understood that an RBD sequence encompasses one or more RBD sequences. An RBD may be linked to or incorporated within an RNA effector molecule. “Linked to” in this context refers to the RBD attached to the N-terminal or C-terminal (if amino acid sequence) or 5′ end or the 3′ end (if nucleotide sequence) of an RNA effector molecule. An RBD linked to an RNA effector molecule may be directly linked with no intervening linker or indirectly linked through an intervening linker to an RNA effector molecule. An intervening linker may be any linker including, but not limited to: a nucleotide sequence (e.g., RNA, DNA, RNA/DNA), a polypeptide sequence that is either cleavable (e.g., by an endonuclease) or non-cleavable, or a disulfide linker. Other linkers may also be used.

An RBD incorporated within an RNA effector molecule may be located anywhere within the RNA effector molecule such that the RNA effector molecule can still perform its function (e.g., detection, RNA editing). In embodiments where an RNA effector molecule is part of CRISPR/Cas nuclease system, an RBD may be located N-terminal to (if amino acid sequence) or 5′ to (if nucleotide sequence), within, or C-terminal to (if amino acid sequence) or 3′ to (if nucleotide sequence) an RNA effector molecule.

An RNA effector molecule of the present disclosure may contain any number of RBDs. In some embodiments, an RNA effector molecule contains 1-50, 1-40, 1-30, 1-20, 1-10, 1-5, 5-50, 5-40, 5-30, 5-20, 5-10, 10-50, 10-40, 10-30, 10-20, 20-50, 20-40, 20-30, 30-50, 30-40, or 40-50 RBDs. When multiple RBDs are present on the same RNA effector molecule, the RBDs may all bind the same RNA aptamer sequence, some may bind different RNA aptamer sequences, or they may all bind to different RNA aptamer sequences. The presence of multiple RBDs on a single RNA effector molecule allows for multiplexing at a target RNA molecule because each RBD will bind a single RNA aptamer sequence.

In some embodiments, an RNA effector molecule comprises an RNA-binding domain (RBD) sequence that specifically binds to an RNA aptamer sequence. Specifically binds refers to preferential binding of a RBD for its corresponding RNA aptamer (e.g., PUF domain→PBS; MCP→MS2; PCP→PP7).

### Kits

The present disclosure, in some embodiments, provides a kit. A kit may comprise, for example, a CRISPR/Cas nuclease gRNA linked to an RNA aptamer sequence and an RNA effector molecule comprising a detectable molecule linked to an RB) sequence that specifically binds to the RNA aptamer sequence. In some embodiments, a kit of the present disclosure further comprises a dCas nuclease. In some embodiments, a catalytically inactive Cas nuclease is a dCas13 nuclease. In some embodiments, an RNA aptamer sequence is a PBS and a RBD is a PUF domain. In some embodiments, an RNA effector molecule is a detectable molecule, such as a fluorescent molecule.

A protein in a kit of the present disclosure may be an isolated protein molecule or a nucleotide sequence that encodes the protein. A nucleotide in a kit of the present disclosure may be an isolated nucleotide molecule or encoded in a larger nucleic acid molecule (e.g., plasmid, vector, etc.).

In addition to the above components, a kit may further include instructions for use of the components and/or practicing the methods. These instructions may be present in the kits in a variety of forms, one or more of which may be present in the kit. One form in which these instructions may be present is as printed information on a suitable medium or substrate, such as a piece or pieces of paper on which the information is printed, in the packaging of the kit, or in a package insert. Yet another means would be a computer readable medium, such as diskette, or CD, on which the information has been recorded. Further, another means by which the instructions may be present is a website address used via the internet to access the information at a removed site.

Components of the kits may be packaged either in aqueous media or in lyophilized form. Kits will generally be packaged to include at least one vial, test tube, flask, bottle, syringe or other container means, into which the described reagents may be placed, and suitably aliquoted. Where additional components are provided, a kit may also generally contain a second, third or other additional container into which such component may be placed.

Kits of the present disclosure may also include a means for containing the reagent containers in close confinement for commercial sale. Such containers may include injection or blow-molded plastic containers into which the desired vials are retained.

### Live Cell Imaging Methods

In some aspects, methods of the present disclosure may be used to image an RNA of interest (or multiple RNAs of interest) in live cells. Imaging an RNA of interest in live cells allows studying RNA dynamics, including but not limited to, RNA editing, transcription, or translocation, in a live cell. The methods may be used to image a single RNA of interest with a single gRNA, to image a single RNA of interest with multiple gRNAs, and to image multiple RNAs of interest with multiple gRNAs.

In some embodiments, live cell imaging methods provided herein may be used to study RNA editing. RNA editing includes, but is not limited to, splicing, methylation, demethylation, degradation, and processing. By studying RNA editing in live cells, intermediates that occur in multi-step processes such as RNA splicing may be visualized in real time. For example, live cell RNA imaging enables the capture and study of dynamic RNA editing states such as formation of the spliceosome during RNA splicing; localization of a methylation or demethylation complex; binding of a degradation molecule; or cleavage prior to 5′ mRNA capping or 3′ poly-adenylation.

For example, live cell RNA imaging may be utilized to visualize intermediates in RNA splicing before mature mRNA is produced. Thus, in some embodiments, the present disclosure provides delivering to a live cell an RNA editing complex comprising a catalytically inactive Cas nuclease (e.g., dCas13), a gRNA comprising (1) a spacer sequence complementary to a noncoding sequence present in a target pre-mRNA molecule and (2) an RNA aptamer sequence, and a fluorescent RNA effector domain fused to an RNA-binding domain that binds the RNA aptamer sequence. The RNA editing complex will assemble at the noncoding target sequence, which will then be visualized in real time using the fluorescent RNA effector domain.

In some embodiments, methods provided herein may be used to study RNA transcription in live cells. In some embodiments, the present disclosure provides delivering to a live cell an RNA editing complex comprising a catalytically inactive Cas nuclease (e.g., dCas13), a gRNA comprising (1) a spacer sequence complementary to a transcription start site sequence and (2) an RNA aptamer sequence, and a fluorescent RNA effector domain fused to an RNA-binding domain that binds the RNA aptamer sequence. The RNA editing complex will assemble at the transcription start site sequence, which will then be visualized in real time using the fluorescent RNA effector domain.

In some embodiments, methods provided herein may be used to study RNA translocation in live cells. For example, nascent transfer RNAs (tRNAs) may be imaged in the nucleus as they are produced and tracked to the cytoplasm of a eukaryotic cell. Thus, in some embodiments, the present disclosure provides delivering to a live cell an RNA editing complex comprising a catalytically inactive Cas nuclease (e.g., dCas13), a gRNA comprising (1) a spacer sequence complementary to a sequence in a nascent tRNA molecule (e.g., D loop, T loop, anticodon loop) and (2) an RNA aptamer sequence, and a fluorescent RNA effector domain fused to an RNA-binding domain that binds the RNA aptamer sequence. The RNA editing complex will assemble at the nascent tRNA sequence, which will then be visualized in real time using the fluorescent RNA effector domain.

In some embodiments, the methods herein comprise imaging a non-repetitive RNA sequence (or multiple non-repetitive RNA sequences) in live cells. Non-repetitive sequences are sequence not repeated in a cell (sequences to which an RNA-editing complex may be bound) or sequences that are not repeated in a single RNA molecule. The ability to image a single non-repetitive sequence in a live cell allows the visualization and capture of dynamic or rare cellular states, such as a disease-causing sequence in a nascent mRNA or an alternatively spliced mature mRNA transcript that is then translated into a mutant protein. By visualizing the non-repetitive sequence, it may be possible to determine the cause of a disease or disorder. For example, imaging a non-repetitive sequence that occurs in the intron of a nascent mRNA transcribed from a gene such as LMNA (Gene ID: 4000) that is subject to alternative splicing may permit distinguishing between various disease states to occur due to alternative splicing, including, but not limited to: Emery-Dreifuss muscular dystrophy, familial partial lipodystrophy, limb girdle muscular dystrophy, dilated cardiomyopathy, Charcot-Marie-Tooth disease, and Hutchinson-Gilford progreria syndrome.

Any other disease or disorder associated with alternative splicing due to a nonrepetitive or repetitive sequence may also be distinguished using methods, kits, and compositions provided herein. Non-limiting examples of such diseases or disorders include: cystic fibrosis, Parkinson's disease, spinal muscular atrophy, myotonic dystrophy type 1, and cancer.

In some embodiments, methods provided herein allow analysis of a pathogenic RNA sequence. Analysis may include imaging to study any pathogenic RNA function, including but not limited to infection, segregation, replication, and packaging. A pathogenic RNA may be derived from any pathogen including, but not limited to, a viral RNA sequence, a bacterial RNA sequence, a protozoal RNA sequence, or a fungal RNA sequence. In some embodiments, a pathogenic RNA sequence is a viral pathogenic RNA sequence. A viral pathogenic RNA sequence may be derived from any virus. Non-limiting examples of viral pathogenic RNA sequences that may be analyzed by methods in the present disclosure include: coronavirus (e.g., SARS-CoV-1, SARS-CoV-2), hepatitis virus (e.g., Hepatitis A, Hepatitis B, Hepatitis C, Hepatitis D, Hepatitis E), influenza virus (e.g., Influenza A, Influenza B, Influenza C, Influenza D), and herpes virus (e.g., herpes simplex 1, herpes simplex 2, varicella zoster, Epstein-Barr, human cytomegalovirus, human herpesvirus 6A, human herpesvirus 6B, human herpesvirus 7, Kaposi's sarcoma-associated herpesvirus).

Thus, in some embodiments, methods provided herein comprise imaging one (or more) non-repetitive sequence by delivering to a live cell an RNA editing complex comprising a catalytically inactive Cas nuclease (e.g., dCas13), a gRNA comprising a spacer sequence complementary to a non-repetitive RNA sequence of interest and an RNA aptamer sequence (e.g., one or more PBS sequence), and an RNA effector molecule comprising an RBD (e.g., one or more PUFs) that specifically binds to the RNA aptamer sequence.

In some aspects, methods provided in the present disclosure allow multiplexed RNA imaging. Multiplexed RNA imaging refers to the assembly of numerous (e.g., more than one) RNA editing complexes in a single live cell. The numerous RNA editing complexes may be assembled on the same RNA molecule, on numerous RNA molecules that exist in the same complex of RNA molecules, or on numerous RNA molecules that exist in different complexes of RNA molecules. For example, a single pre-mRNA molecule may be subject to multiplex imaging in its noncoding and coding regions simultaneously to visualize pre-mRNA splicing. Multiple pre-mRNAs that are polycistronic (transcribed in tandem and cut apart by splicing factors) may be subject to multiplex imaging in their noncoding regions simultaneously. Multiple RNA molecules that exist in separate complexes (e.g., mRNAs and ribosomal RNAs or transfer RNAs) may be subject to multiplex imaging simultaneously.

Multiplexed imaging may be accomplished using multiple single gRNAs that each contain a spacer region that is complementary to a unique single target sequence and a unique single RNA aptamer or using a single gRNA that contains multiple spacer regions and RNA aptamers that are each complementary to a single RNA target sequence and RBD, respectively.

In some embodiments, methods comprise, for example, delivering to a live cell (a) catalytically inactive CRISPR/Cas nuclease (e.g., dCas13), (b) a Cas gRNA (e.g., Cas13 gRNA) comprising an RNA aptamer sequence (e.g., one or more PBS sequences), and an RNA effector molecule comprising a detectable molecule and an RBD (e.g., one or more PUF domains) that specifically binds to the RNA aptamer sequence and imaging the detectable molecule.

Thus, in some embodiments, the present disclosure provides a multiplex live cell imaging method comprising transfecting a cell with a first Cas13 gRNA linked to a first RNA aptamer sequence and a first RNA effector molecule linked to a first RNA-binding domain (RBD) sequence that specifically binds to the first RNA aptamer sequence; and a second Cas13 gRNA linked to a second RNA aptamer sequence and a second RNA effector molecule linked to a second RBD sequence that specifically binds to the second RNA aptamer sequence. In some embodiments, a first and a second RNA aptamer sequence are PBSs and a first and a second RBD sequence are PUFs.

In some aspects, methods provided herein are used to image multiple RNA foci in live cells. An RNA focus may contain a single RNA molecule or multiple RNA molecules (e.g., tens, hundreds). For example, the methods may be used to image 2-100, 2-75, 2-50, 2-25, 2-15, 2-10, 5-100, 5-75, 5-50, 5-25, 5-15, 5-10, 10-100, 10-75, 10-50, 10-25, or 10-15 RNA foci in live cells. In some embodiments, the methods may be used to image 2, 3, 4, 5, 6, 7, 8, 9, 10, 11, 12, 13, 14, 15, or more RNA foci in live cells. Thus, in some embodiments, the live cells are transfected with 2-100, 2-75, 2-50, 2-25, 2-15, 2-10, 5-100, 5-75, 5-50, 5-25, 5-15, 5-10, 10-100, 10-75, 10-50, 10-25, or 10-15 gRNAs (or nucleic acids encoding the gRNAs). For example, live cells herein may be transfected with 2, 3, 4, 5, 6, 7, 8, 9, 10, 11, 12, 13, 14, 15, or more gRNAs. Transfection may be by any method. Non-limiting methods of transfection include: electroporation, calcium phosphate, liposome, and viral (e.g., lentiviral, adenoviral, adeno-associated virus, retroviral) transfection

Imaging may occur 12-96 hours post-transfection. For example, imaging may occur 12, 24, 36, 48, 60, 72, 84, or 96 hours after transfection. As another example, imaging may occur 12-24, 12-48, 12-72, 24-48, 24-72, or 48-72 hours post-transfection. Imaging may occur for less than 1, 2, 3, 4, 5, 6, 7, 8, 9, 10, 11, 12, 13, 14, or 15 minutes. In some embodiments, images are taken at certain time points, for example, every 1, 2, 3, 4, 5, 6, 7, 8, 9, 10, 15, 20, 25, 30, 35, 40, 45, 50, 55, or 60 seconds. In some embodiments, images are taken every 1, 2, 3, 4, 5, 6, 7, 8, 9, 10, 15, 20, 25, 30, 35, 40, 45, 50, 55, or 60 minutes. In some embodiments, imaging takes place over a period of 1, 2, 3, 4, 5, 6, 7, 8, 9, 10, 11, 12, 14, 16, 18, 20, 24, 36, 48, 60, or 72 hours. For example, images may be captured in 30 minutes for 5, 6, 7, 8, 9, 10, 11, 12, 13, 14, 15, 16, 17, 18, 19, or 20 hours.

Imaging may be accomplished by any method. The method of imaging selected depends on the detectable molecule used. For example, fluorescent microscopy (e.g., confocal fluorescent microscopy) can be used to examine the live cell populations when a fluorescent detectable molecule is used.

A cell (one or more) may be any cell that comprises an RNA-editing complex. Any cell that contains RNA may be imaged with methods provided in the present disclosure. Non-limiting examples of a cell that may be imaged include: mammalian, plant, bacterial, protozoan, amphibian, insect, and reptilian cells. In some embodiments, a cell is a mammalian cell. A mammalian cell may be from any mammal including, but not limited to, a human, a mouse, a rat, a non-human primate, a dog, a cat, and a pig. A cell may be any type of cell including, but not limited to neurons, fibroblasts, epithelial cells, muscle cells, lymphocytes, macrophages, and endothelial cells.

### Other Methods

In some aspects, methods of the present disclosure may be used to edit RNA. As described above, editing RNA may be by any method including, but not limited to: splicing, methylation (or demethylation), targeting, or processing the RNA.

In some embodiments, methods provided herein allow splicing of a target RNA. Splicing may occur when an RNA editing complex comprises an RNA effector that is an RNA splicing factor. For example, an RNA effector that allows splicing of a target RNA may be RBFOX1, U2 small nuclear RNA auxiliary factor 1 (U2AF35), U2AF2 (U2AF65), splicing factor 1 (SF1), U1 small nuclear ribonucleoprotein (snRNP), U2 snRNP, U4 snRNP, U5 snRNP, U6 snRNP, U11, U12, U4atac, or U6atac. It should be understood that multiple RNA splicing factor effectors may be present at a target RNA in a process known as multiplexed RNA splicing.

In some embodiments, methods provided herein allow methylation or demethylation of a target RNA. Methylation and demethylation may occur when an RNA editing complex comprises an RNA effector that is an RNA methylation protein or an RNA demethylation protein. For example, an RNA effector that allows methylation or demethylation of a target RNA may be METTL3, METTL14, WTAP, VIRMA, ZC3H13, RBM15, RBM15B, HAKAI, METTL16, METTL5, FTO, or ALKBH5. It should be understood that multiple RNA methylation or demethylation effectors may be present a target RNA in a process known as multiplexed RNA methylation or multiplexed RNA demethylation.

In some embodiments, methods provided herein allow degradation of a target RNA molecule. Degradation may occur when an RNA editing complex comprises an RNA effector that is an RNA degradation molecule. For example, an RNA effector that allows degradation of a target RNA may be a protein including Rnt1p; a chimera including ribonuclease targeting chimeras (RIBOTACs) or (2′-5′)oligoadenylate antisense chimera; or a small molecule including Targapremir-210 (TGP-210). It should be understood that multiple RNA degradation molecules may be present at a target RNA in a process known as multiplexed RNA degradation.

In some embodiments, methods provided herein allow processing of a target RNA molecule. Processing may occur when an RNA editing complexes comprises an RNA effector domain that is an RNA processing molecule. For example, an RNA effector that allows processing of a target RNA may be RNA triphosphatase, guanosyl transferase, guanine-N⁷-methyltransferase, cleavage and polyadenylation specificity factor, cleavage stimulation factor, cleavage factor 1, polyadenylate polymerase, or cleavage and polyadenylation specificity factor 73. It should be understood that multiple RNA processing molecules may be present at a target RNA in a process known as multiplexed RNA processing.

### Compositions

Some aspects of the present disclosure provide a composition comprising an RNA editing complex. In some embodiments, an RNA editing complex in a composition comprising a catalytically inactive Cas13 (dCas13) nuclease, a Cas13 gRNA comprising an RNA aptamer sequence, and an RNA effector molecule comprising (i) a detectable molecule and an RNA binding domain (RBD).

In some embodiments, an RNA editing complex comprises 2-100, 2-75, 2-50, 2-25, 2-15, 2-10, 5-100, 5-75, 5-50, 5-25, 5-15, 5-10, 10-100, 10-75, 10-50, 10-25, or 10-15 Cas13 gRNAs comprising RNA aptamer sequences and 2-100, 2-75, 2-50, 2-25, 2-15, 2-10, 5-100, 5-75, 5-50, 5-25, 5-15, 5-10, 10-100, 10-75, 10-50, 10-25, or 10-15 RNA effector molecules comprising RNA binding domains (RBDs) that bind the RNA aptamer sequences.

In some embodiments, the RNA aptamer sequences are Pumilio binding sequences (PBSs) and the RBDs are Pumilio-FBF (PUF) domains. A PBS sequence may be any sequence described herein, and a PUF domain may be any PUF domain described herein. Thus, in some embodiments, an RNA editing complex comprises 2-100, 2-75, 2-50, 2-25, 2-15, 2-10, 5-100, 5-75, 5-50, 5-25, 5-15, 5-10, 10-100, 10-75, 10-50, 10-25, or 10-15 Cas13 gRNAs comprising PBSs and 2-100, 2-75, 2-50, 2-25, 2-15, 2-10, 5-100, 5-75, 5-50, 5-25, 5-15, 5-10, 10-100, 10-75, 10-50, 10-25, or 10-15 RNA effector molecules comprising PUFs (RBDs) that bind the PBSs.

In some embodiments, a composition comprises an excipient. Non-limiting examples of excipients include antiadherents (e.g., magnesium stearate), binders (e.g., sucrose, lactose, starches, cellulose, microcrystalline cellulose, hydroxypropyl cellulose), sugar alcohols (e.g., xylitol, sorbitol, mannitol), protein (e.g., gelatin), synthetic polymers (e.g., polyvinylpyrrolidone, polyethylene glycol), coatings (e.g., hydroxyproypl methylcellulose, shellac, corn protein zein); disintegrants (e.g., crosslinked sodium caroxymethyl cellulose), starch (e.g., glycolate), glidants (e.g., silica gel, fumed silica, talc, magnesium carbonate), preservatives (e.g., vitamin A, vitamin E, vitamin C, retinyl palmitate, selenium, cysteine, methionine, citric acid, sodium citrate, methyl paraben, propyl paraben), and vehicles (e.g., petrolatum, dimethyl sulfoxide, mineral oil).

### Additional Embodiments

Additional embodiments are encompassed by the following numbered paragraphs:
1. A ribonucleic acid (RNA)-editing complex, comprising:
   a catalytically inactive Cas13 (dCas13) nuclease;
   a Cas13 guide RNA (gRNA) comprising an RNA aptamer sequence; and
   an RNA effector molecule comprising an RNA-binding domain (RBD) sequence that specifically binds to the RNA aptamer sequence.
2. The RNA-editing complex of paragraph 1, wherein the dCas13 nuclease is pre-crRNA processing deficient.
3. The RNA-editing complex of paragraph 1 or 2, wherein the dCas13 nuclease is a dCas13b nuclease.
4. The RNA-editing complex of any one of the preceding paragraphs, wherein the dCas13 nuclease is a Prevotella dCas13 nuclease.
5. The RNA-editing complex of paragraph 4, wherein the Prevotella dCas13 nuclease is a Prevotella sp. P5-125 dCas13 nuclease (PspdCas13).
6. The RNA-editing complex of any one of paragraphs 2-5, wherein the dCas13 nuclease comprises a mutation at one or more position(s) corresponding to amino acid positions 367-370 of the amino acid sequence of SEQ ID NO: 1.
7. The RNA-editing complex of paragraph 6, wherein the mutation at one or more position(s) corresponding to amino acid positions 367-370 of the amino acid sequence of SEQ ID NO: 1 is mutated to a nonpolar neutral amino acid.
8. The RNA-editing complex of paragraph 7, wherein the nonpolar neutral amino acid is alanine.
9. The RNA-editing complex of any one of the preceding paragraphs, wherein the RNA aptamer is selected from a Pumilio aptamer sequence, an MS2 aptamer sequence, and a PP7 aptamer sequence.
10. The RNA-editing complex of paragraph 9, wherein the RNA aptamer sequence is a Pumilio aptamer sequence and the RBD sequence is a Pumilio binding domain sequence.
11. The RNA-editing complex of paragraph 9, wherein the RNA aptamer sequence is an MS2 aptamer sequence and the RBD sequence is an MS2 coat protein (MCP) sequence.
12. The RNA-editing complex of paragraph 9, wherein the RNA aptamer sequence is a PP7 aptamer sequence and the RBD sequence is a PP7 coat protein (PCP) sequence.
13. The RNA-editing complex of any one of the preceding paragraphs, wherein the RNA effector molecule is selected from detectable molecules, RNA splicing factors, RNA methylation or demethylation proteins, RNA degradation molecules, and RNA processing molecules.
14. A kit, comprising:
   a Cas13 guide RNA (gRNA) linked to an RNA aptamer sequence; and
   an RNA effector molecule linked to an RNA-binding domain (RBD) sequence that specifically binds to the RNA aptamer sequence.
15. The kit of paragraph 14 further comprising a catalytically inactive Cas13 (dCas13) nuclease.
16. A method, comprising transfecting a cell with a Cas13 guide RNA (gRNA) linked to an RNA aptamer sequence and an RNA effector molecule linked to an RNA-binding domain (RBD) sequence that specifically binds to the RNA aptamer sequence.
17. The method of paragraph 16 further comprising transfecting the cell with a catalytically inactive Cas13 (dCas13) nuclease.
18. The method of paragraph 16 or 17, wherein the cell comprises an RNA of interest and the gRNA specifically binds to the RNA of interest.
19. The method of paragraph 18 further comprising incubating the cell to modify the RNA of interest.
20. A method, comprising transfecting a cell with:
   a first Cas13 guide RNA (gRNA) linked to a first RNA aptamer sequence and a first RNA effector molecule linked to a first RNA-binding domain (RBD) sequence that specifically binds to the first RNA aptamer sequence; and
   a second Cas13 gRNA linked to a second RNA aptamer sequence and a second RNA effector molecule linked to a second RBD sequence that specifically binds to the second RNA aptamer sequence.
21. The method of cl aim 20 further comprising transfecting the cell with a catalytically inactive Cas13 (dCas13) nuclease.
22. The method of paragraph 20 or 21, wherein the cell comprises a first RNA of interest and a second RNA of interest, the first Cas13 gRNA specifically binds to the first RNA of interest, and the second Cas13 gRNA specifically binds to the first second of interest.
23. The method of paragraph 22 further comprising incubating the cell to modify the first RNA of interest and the second RNA of interest.

### EXAMPLES

### Example 1: Multiplexed RNA Targeting System

The gRNA of Cas13 was tagged with different RNA aptamers designed to recruit distinct effectors fused with Cas13 cognate RNA binding domains (RBDs, e.g., PUF/MCP/PCP) to execute different RNA editing functions (FIG. 1B). By pairing the RNA aptamers on the target-specific gRNAs with cognate Cas13 RBD-fused functional effectors, it was possible to modulate different RNA processes and achieve multi-color imaging of multiple RNAs in the same cell with a single dCas13.

Because Cas13 proteins are known to process polycistronic pre-crRNA by cleaving between the direct repeats (DRs) and target spacers, wild-type Cas13 may cleave away the aptamers appended to the gRNA, potentially necessitating the inactivation of the crRNA processing activity of Cas13 in the context of the present technology. In Prevotella buccae Cas13b (PbuCas13b), the residue K393 in its lid domain was identified to be required for processing of pre-crRNAs (Slaymaker et al., Cell Reports, 2019). Alignment between Prevotella sp. P5-125 (PspCas13b) and PbuCas13b revealed amino acids 367-370 (KADK) of PspCas13b (SEQ ID NO: 1) may possess a similar crRNA processing activity (FIG. 1C). To ensure the RNA aptamer array of a gRNA is not cleaved by PspCas13b, the charged amino acids 367-370 (KADK) were mutated to alanine to create the dpspCas13b(AAAA) mutant (SEQ ID NO: 2).

### Example 2: Multiplex RNA Targeting System-Mediated RNA Splicing Modulation

Spinal muscular atrophy (SMA) is a hereditary neuronal disease caused by the defect in survival motor neuron 1 (SMN1). The inclusion of exon 7 in the mRNA of SMN2, the homolog of SMN1, is able to restore SMN protein levels and rescue SMA symptoms.

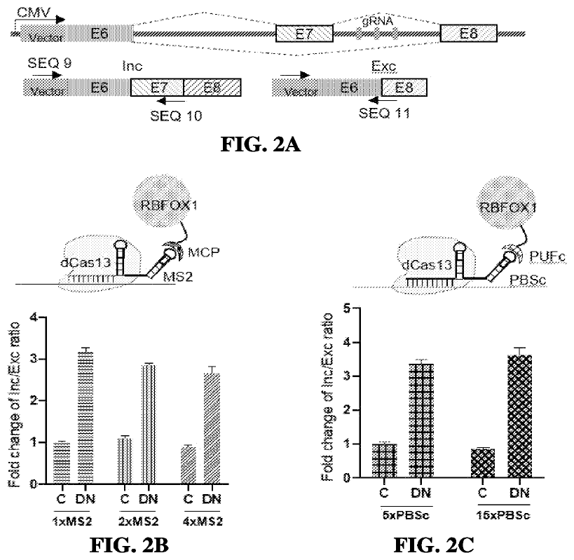

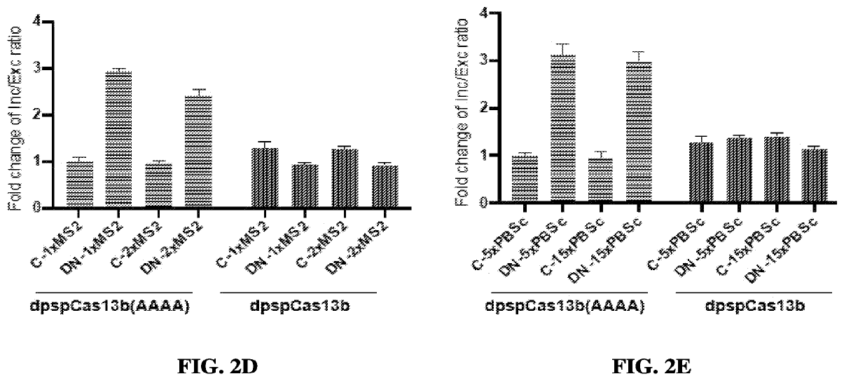

To induce the inclusion of SMN2 exon 7, three gRNAs (SEQ ID NOs: 21-23) were designed complementary to sequences in the intron downstream of exon 7 called ‘DN’ for targeting as previously reported (e.g., Du et al., Nat. Commun., 2020) and tagged with different numbers of MS2 and PBSc sequences. Then, functional RNA processing modules were constructed by replacing the RRM region (118-189) in splicing factor RBFOX1 with MCP and PUFc sequences to produce MCP-RBFOX1 (SEQ ID NO: 3) and PUFc-RBFOX1 (SEQ ID NO: 4), respectively. Two pairs of primers were also designed to amplify the pCI-SMN2 (containing the splicing minigene) transcripts with inclusion and exclusion of exon 7, respectively, and the ratio of inclusion/exclusion was used to estimate the alternative splicing efficacy (FIG. 2A; SEQ ID NOs: 9-12). When HEK293T cell were co-transfected with the pCI-SMN2 splicing minigene reporter plasmid (SEQ ID NO: 8) and the alternative splicing components 1 (“RAS 1”: dpspCas13b(AAAA), MCP-RBFOX1, gRNAs tagged with MS2), a significant increase of SMN2 exon 7 inclusion was observed in cells transfected with the on-target gRNAs compared with those transfected with a control non-targeting gRNA (SEQ ID NO: 20) (FIG. 2B). Increasing the copy number of MS2 sequences did not increase the efficacy of inclusion of SMN2 exon 7 by RAS1. Similarly, the alternative splicing components 2 (“RAS2”: dpspCas13b(AAAA). PUFc-RBFOX1, gRNAs tagged with PBSc) induced SMN2 exon 7 inclusion 3- to 4-fold over control levels, and there was no significant difference in SMN2 exon inclusion between RAS2 with 5 copies and 15 copies of PBSc (FIG. 2C). To confirm whether the dPspCas13b(AAAA) mutation is required for RAS1 and RAS2 activity, RAS complexes with dPspCas13b(AAAA) or crRNA-processing-active dpspCas13b were compared for the induction of SMN2 exon 7 inclusion. RAS complexes with crRNA processing activity did not induce splicing activation, confirming the requirement of the dPspCas13b(AAAA) mutation for the function of tripartite complexes (FIGS. 2D-2E).

### Example 3: Design of RNA Scaffold

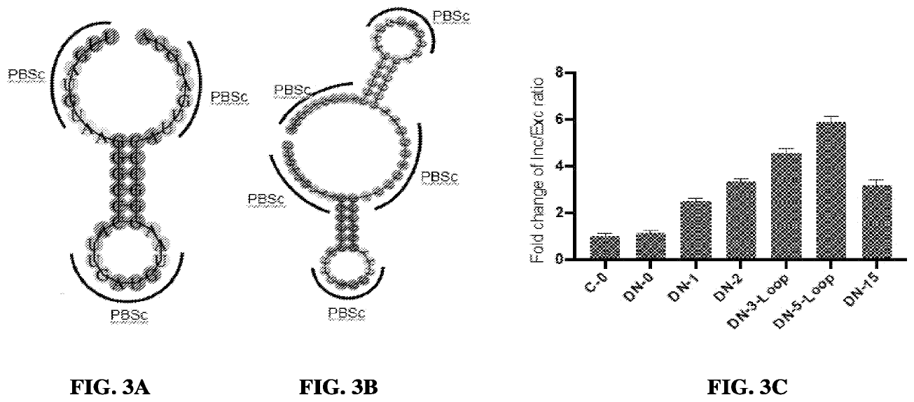

Increasing the copy number of MS2 and PBSc sequences on gRNA number did not improve the efficacy of RAS1 and RAS2. This may be because the design of the PBS array with GCC spacing is suboptimal in the context of gRNA. The RNA scaffold was edited by stabilizing its structure with stem loops. Taking PBSc as an example, a stem loop structure was added between two PBSc to generate the 3 copies of PBSc with one stem loop (“3-Loop”, FIG. 3A, SEQ ID NO: 40) and 5 copies of PBSc with two stem loops (“5-Loop”, FIG. 3B, SEQ ID NO: 42). Strikingly, splicing modulation was significantly enhanced with the gRNA tagged by loop-stabilized PBSc (FIG. 3C). gRNAs with 3 or 5 copies of loop-stabilized PBSc surpassed gRNAs with 15 copies of non-stabilized PBSc in splicing activation, demonstrating the optimization of aptamer array through RNA structure-guided design.

### Example 4: Orthogonality of gRNA-Aptamer:RBD-Effector Pairs

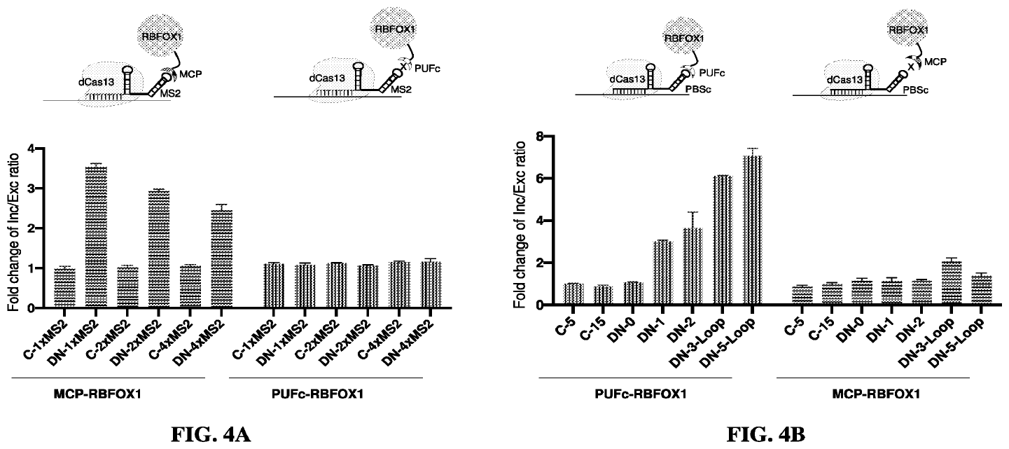

For multiplexed RNA editing, different aptamer systems should act independently, and the recognition between RNA scaffolds and RBDs must be specific. To test whether there is crosstalk between aptamer systems, MS2-tagged gRNAs with PUFc-fused RBFOX1 and the PBSc-tagged gRNAs with MCP-fused RBFOX1 were co-transfected and the inclusion of exon7 in SMN2 was measured using a splicing reporter. Significantly, the unmatched gRNA-aptamer and RBD-effector pairs showed no effect on the alternative splicing of SMN2 exon 7 (FIGS. 4A-4B), demonstrating that gRNA-aptamer:RBD-effector pairing are orthogonal in the context of the present disclosure and establishing the fundamental mechanism for functional multiplexing.

### Example 5: Site-Specific RNA m6A Modification

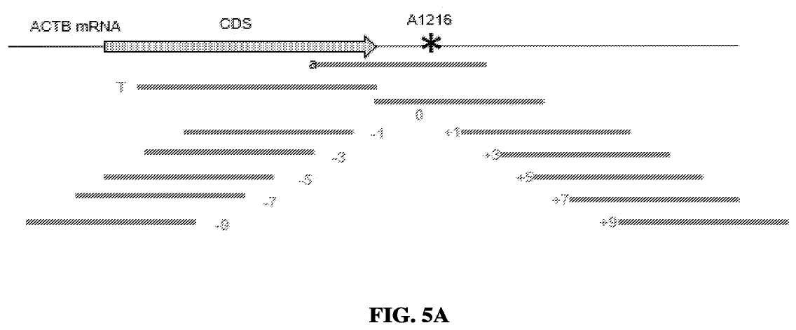

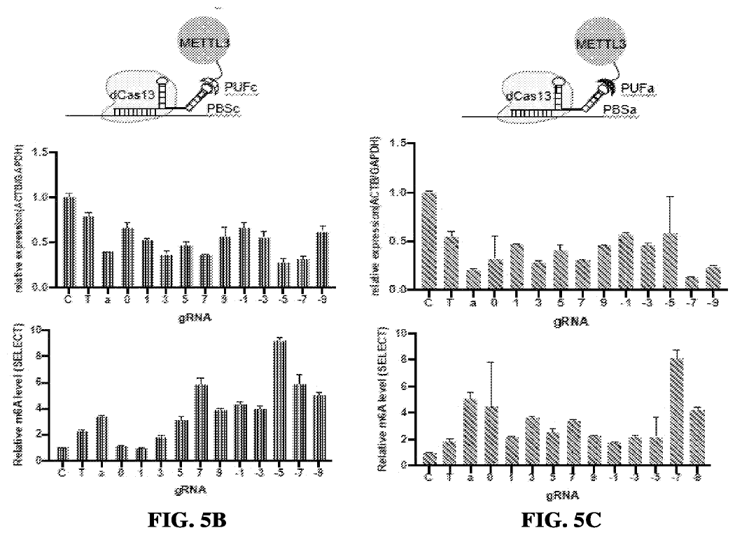

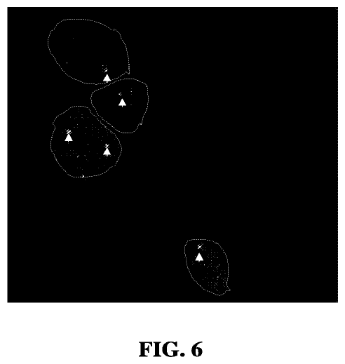

The multiplex RNA targeting system was tested on site-specific RNA m6A modification. Given that A1216 in ACTB mRNA is known to be methylated in multiple cell lines and the m6A modification at A1216 can reduce the RNA stability of ACTB, it was chosen as the first target and the mRNA level was used as the preliminary readout. The catalytic domain of RNA methyltransferase METTL3 (M3, 273-580) was fused to two different PUF variants, PUFa (SEQ ID NO: 6) and PUFc (SEQ ID NO: 5). For the gRNAs targeting to ACTB, two previously reported gRNAs were tested (SEQ ID NOs: 24-25) (e.g., Liu et al., Nat. Chem. Biol, 2019; Wilson et al., Nat. Biotechnol., 2020), and more gRNAs that shift every two nucleotides from the A1216 site at both directions were screened (FIG. 5A, SEQ ID NOs: 26-36). HEK293T cells transfected with PUFa-M3/PUFc-M3 showed a lower expression level of ACTB with the on-target gRNA compared with non-target control gRNA, which indicates the successful deposition of m6A modification at A1216 site (FIGS. 5B-5C). Furthermore, the editing levels of m6A were confirmed by SELECT PCR (Xiao et al., Angewandte Chemie International Edition, 2018), which detects and quantifies m6A at the single base level and found that the A1216 m6A level was increased in most of the on-target gRNA, especially for the gRNAs targeting the upstream of A1216 site (FIGS. 5B-5C). Taken together, these findings show that multiplex RNA targeting system induces site-specific RNA m6A modification on endogenous transcripts.

### Example 6: RNA Live-Cell Imaging of Nonrepetitive Sequence

Both aptamers and the CRISPR/Cas system have been used for live-cell RNA imaging. However, the insertion of aptamers like MS2 may disrupt the localization and degradation of target RNA while the CRISPR/Cas system only works for RNA granules and endogenous RNAs with multiple repeated sequences (Yang et al., Mol. Cell, 2019). To test whether the system provided herein overcomes the barrier of non-repetitive RNA sequence labeling, a gRNA was designed (SEQ ID NO: 37) targeting the intron of LMNA gene with 15 copies of PBSc motifs to image its nascent transcripts. Significantly, HEK293T cells co-transfected with dpspCas13b(AAAA), Clover-NLS-PUFc (SEQ ID NO: 7) and the gRNA (SEQ ID NO: 37) with 15×PBSc showed bright GFP foci in the nuclei, corresponding to the nascent LMNA transcripts at the LMNA locus (FIG. 6).

SEQUENCES
amino acid sequence of dpspCas13b
>SEQ ID NO: 1
MNIPALVENQKKYFGTYSVMAMLNAQTVLDHIQKVADIEG
EQNENNENLWFHPVMSHLYNAKNGYDKQPEKTMFIIERLQ
SYFPFLKIMAENQREYSNGKYKQNRVEVNSNDIFEVLKRA
FGVLKMYRDLTNAYKTYEEKLNDGCEFLISTEQPLSGMIN
NYYTVALRNMNERYGYKTEDLAFIQDKRFKFVKDAYGKKK
SQVNTGFFLSLQDYNGDTQKKLHLSGVGIALLICLFLDKQ
YINIFLSRLPIFSSYNAQSEERRIIIRSFGINSIKLPKDR
IHSEKSNKSVAMDMLNEVKRCPDELFTTLSAEKQSRFRII
SDDHNEVLMKRSSDRFVPLLLQYIDYGKLFDHIRFHVNMG
KLRYLLKADKTCIDGQTRVRVIEQPLNGFGRLEEAETMRK
QENGTFGNSGIRIRDFENMKRDDANPANYPYIVDTYTHYI
LENNKVEMFINDKEDSAPLLPVIEDDRYVVKTIPSCRMST
LEIPAMAFHMFLFGSKKTEKLIVDVHNRYKRLFQAMQKEE
VTAENIASFGIAESDLPQKILDLISGNAHGKDVDAFIRLT
VDDMLTDTERRIKRFKDDRKSIRSADNKMGKRGFKQISTG
KLADFLAKDIVLFQPSVNDGENKITGLNYRIMQSAIAVYD
SGDDYEAKQQFKLMFEKARLIGKGTTEPHPFLYKVFARSI
PANAVEFYERYLIERKFYLTGLSNEIKKGNRVDVPFIRRD
QNKWKTPAMKILGRIYSEDLPVELPRQMEDNEIKSHLKSL
PQMEGIDENNANVTYLIAEYMKRVLDDDFQTFYQWNRNYR
YMDMLKGEYDRKGSLQHCFTSVEEREGLWKERASRTERYR
KQASNKIRSNRQMRNASSEEIETILDKRLSNSRNEYQKSE
KVIRRYRVQDALLFLLAKKTLTELADFDGEREKLKEIMPD
AEKGILSEIMPMSFTFEKGGKKYTITSEGMKLKNYGDFFV
LASDKRIGNLLELVGSDIVSKEDIMEEFNKYDQCRPEISS
IVFNLEKWAFDTYPELSARVDREEKVDFKSILKILLNNKN
INKEQSDILRKIRNAFDANNYPDKGVVEIKALPEIAMSIK
KAFGEYAIMK,
amino acid sequence of dpspCas13b (AAAA)
>SEQ ID NO: 2
MNIPALVENQKKYFGTYSVMAMLNAQTVLDHIQKVADIEG
EQNENNENLWFHPVMSHLYNAKNGYDKQPEKTMFIIERLQ
SYFPFLKIMAENQREYSNGKYKQNRVEVNSNDIFEVLKRA
FGVLKMYRDLTNAYKTYEEKLNDGCEFLTSTEQPLSGMIN
NYYTVALRNMNERYGYKTEDLAFIQDKRFKFVKDAYGKKK
SQVNTGFFLSLQDYNGDTQKKLHLSGVGIALLICLFLDKQ
YINIFLSRLPIFSSYNAQSEERRIIIRSFGINSIKLPKDR
IHSEKSNKSVAMDMLNEVKRCPDELFTTLSAEKQSRFRII
SDDHNEVLMKRSSDRFVPLLLQYIDYGKLFDHIRFHVNMG
KLRYLLAAAATCIDGQTRVRVIEQPLNGFGRLEEAETMRK
QENGTFGNSGIRIRDFENMKRDDANPANYPYIVDTYTHYI
LENNKVEMFINDKEDSAPLLPVIEDDRYVVKTIPSCRMST
LEIPAMAFHMFLFGSKKTEKLIVDVHNRYKRLFQAMQKEE
VTAENIASFGIAESDLPQKILDLISGNAHGKDVDAFIRLT
VDDMLTDTERRIKRFKDDRKSIRSADNKMGKRGFKQISTG
KLADFLAKDIVLFQPSVNDGENKITGLNYRIMQSAIAVYD
SGDDYEAKQQFKLMFEKARLIGKGTTEPHPFLYKVFARSI
PANAVEFYERYLIERKFYLTGLSNEIKKGNRVDVPFIRRD
QNKWKTPAMKTLGRIYSEDLPVELPRQMEDNEIKSHLKSL
PQMEGIDENNANVTYLIAEYMKRVLDDDFQTFYQWNRNYR
YMDMLKGEYDRKGSLQHCFTSVEEREGLWKERASRTERYR
KQASNKIRSNRQMRNASSEEIETILDKRLSNSRNEYQKSE
KVIRRYRVQDALLFLLAKKTLTELADFDGEREKLKEIMPD
AEKGILSEIMPMSFTFEKGGKKYTITSEGMKLKNYGDFFV
LASDKRIGNLLELVGSDIVSKEDIMEEFNKYDQCRPEISS
IVFNLEKWAFDTYPELSARVDREEKVDFKSILKILLNNKN
INKEQSDILRKIRNAFDANNYPDKGVVEIKALPEIAMSIK
KAFGEYAIMK,
amino acid sequence of NLS-MCP-RBFQX1
>SEQ ID NO: 3
MNCEREQLRGNQEAAAAPDTMAQPYASAQFAPPQNGIPAE
YTAPHPHPAPEYTGQTTVPEHTLNLYPPAQTHSEQSPADT
SAQTVSGTATQTDDAAPTDGQPQTQPSENTENKSQPKGGG
GSGRAMASNFTQFVLVDNGGTGDVTVAPSNFANGVAEWIS
SNSRSQAYKVTCSVRQSSAQKRKYTIKVEVPKVATQTVGG
VELPVAAWRSYLNMELTIPIFATNSDCELIVKAMQGLLKD
GNPIPSAIAANSGIYSAGGRGGGGSGGGGSGGGGSGPANA
TARVMTNKKTVNPYTNGWKLNPVVGAVYSPEFYAGTVLLC
QANQEGSSMYSAPSSLVYTSAMPGFPYPAATAAAAYRGAH
LRGRGRTVYNTFRAAAPPPPIPAYGGVVYQDGFYGADIYG
GYAAYRYAQPTPATAAAYSDSYGRVYAADPYHHALAPAPT
YGVGAMNAFAPLTDAKTRSHADDVGLVLSSLQASIYRGGY
NRFAPY,
amino acid sequence of NLS-PUFc-RBFQX1
>SEQ ID NO: 4
MNCEREQLRGNQEAAAAPDTMAQPYASAQFAPPQNGIPAE
YTAPHPHPAPEYTGQTTVPEHTLNLYPPAQTHSEQSPADT
SAQTVSGTATQTDDAAPTDGQPQTQPSENTENKSQPKGGG
GSGRAGILPPKKKRKVSRGRSRLLEDFRNNRYPNLQLREI
AGHIMEFSQDQHGSRFIQLKLERATPAERQLVFNEILQAA
YQLMVDVFGNYVIQKFFEFGSLEQKLALAERIRGHVLSLA
LQMYGSRVIEKALEFIPSDQQNEMVRELDGHVLKCVKDQN
GNHVVQKCIECVQPQSLQFIIDAFKGQVFALSTHPYGCRV
IQRILEHCLPDQTLPILEELHQHTEQLVQDQYGSYVIEHV
LEHGRPEDKSKIVAEIRGNVLVLSQHKFANNVVQKCVTHA
SRTERAVLIDEVCTMNDGPHSALYTMMKDQYANYVVQKMI
DVAEPGQRKIVMHKIRPHIATLRKYTYGKHILAKLEKYYM
KNGVDLGDPKKKRKVDPKKKRKVGGRGGGGSGGGGSGGGG
SGPANATARVMTNKKTVNPYTNGWKLNPVVGAVYSPEFYA
GTVLLCQANQEGSSMYSAPSSLVYTSAMPGFPYPAATAAA
AYRGAHLRGRGRTVYNTFRAAAPPPPIPAYGGVVYQDGFY
GADIYGGYAAYRYAQPTPATAAAYSDSYGRVYAADPYHHA
LAPAPTYGVGAMNAFAPLTDAKTRSHADDVGLVLSSLQAS
IYRGGYNRFAPY,
amino acid sequence of NLS-PUFc-METTL3
>SEQ ID NO: 5
MDYKDHDGDYKDHDIDYKDDDDKIDGGGGSDPKKKRKVDP
KKKRKVDPKKKRKVGSTGSRNDGGGGSGGGGSGGGGSGRA
GILPPKKKRKVSRGRSRLLEDFRNNRYPNLQLREIAGHIM
EFSQDQHGSRFIQLKLERATPAERQLVFNEILQAAYQLMV
DVFGNYVIQKFFEFGSLEQKLALAERIRGHVLSLALQMYG
SRVIEKALEFIPSDQQNEMVRELDGHVLKCVKDQNGNHVV
QKCIECVQPQSLQFIIDAFKGQVFALSTHPYGCRVIQRIL
EHCLPDQTLPILEELHQHTEQLVQDQYGSYVIEHVLEHGR
PEDKSKIVAEIRGNVLVLSQHKFANNVVQKCVTHASRTER
AVLIDEVCTMNDGPHSALYTMMKDQYANYVVQKMIDVAEP
GQRKIVMHKIRPHIATLRKYTYGKHILAKLEKYYMKNGVD
LGDPKKKRKVDPKKKRKVGGRGGGGSGGGGSGGGGSGPAQ
EFCDYGTKEECMKASDADRPCRKLHFRRIINKHTDESLGD
CSFLNTCFHMDTCKYVHYEIDACMDSEAPGSKDHTPSQEL
ALTQSVGGDSSADRLFPPQWICCDIRYLDVSILGKFAVVM
ADPPWDIHMELPYGTLTDDEMRRLNIPVLQDDGFLFLWVT
GRAMELGRECLNLWGYERVDEIIWVKTNQLQRIIRTGRTG
HWLNHGKEHCLVGVKGNPQGFNQGLDCDVIVAEVRSTSHK
PDEIYGMIERLSPGTRKIELFGRPHNVQPNWITLGNQLDG
IHLLDPDVVARFKQRYPDGIISKPKNL,
amino acid sequence of NLS-PUFa-METTL3
>SEQ ID NO: 6
MDYKDHDGDYKDHDIDYKDDDDKIDGGGGSDPKKKRKVDP
KKKRKVDPKKKRKVGSTGSRNDGGGGSGGGGSGGGGSGRA
SRGRSRLLEDFRNNRYPNLQLREIAGHIMEFSQDQHGSRF
IQLKLERATPAERQLVFNEILQAAYQLMVDVFGNYVIQKF
FEFGSLEQKLALAERIRGHVLSLALQMYGSRVIEKALEFI
PSDQQNEMVRELDGHVLKCVKDQNGNHVVQKCIECVQPQS
LQFIIDAFKGQVFALSTHPYGCRVIQRILEHCLPDQTLPI
LEELHQHTEQLVQDQYGNYVIQHVLEHGRPEDKSKIVAEI
RGNVLVLSQHKFASNVVEKCVTHASRTERAVLIDEVCTMN
DGPHSALYTMMKDQYANYVVQKMIDVAEPGQRKIVMHKIR
PHIATLRKYTYGKHILAKLEKYYMKNGVDLGGGRGGGGSG
GGGSGGGGSGPAQEFCDYGTKEECMKASDADRPCRKLHFR
RIINKHTDESLGDCSFLNTCFHMDTCKYVHYEIDACMDSE
APGSKDHTPSQELALTQSVGGDSSADRLFPPQWICCDIRY
LDVSILGKFAVVMADPPWDIHMELPYGTLTDDEMRRLNIP
VLQDDGFLFLWVTGRAMELGRECLNLWGYERVDEIIWVKT
NQLQRIIRTGRTGHWLNHGKEHCLVGVKGNPQGFNQGLDC
DVIVAEVRSTSHKPDEIYGMIERLSPGTRKIELFGRPHNV
QPNWITLGNQLDGIHLLDPDVVARFKQRYPDGIISKPKNL,
amino acid sequence of Clover-NLS-PUFc
>SEQ ID NO: 7
MVSKGEELFTGVVPILVELDGDVNGHKFSVRGEGEGDATN
GKLTLKFICTTGKLPVPWPTLVTTFGYGVACFSRYPDHMK
QHDFFKSAMPEGYVQERTISFKDDGTYKTRAEVKFEGDTL
VNRIELKGIDFKEDGNILGHKLEYNFNSHNVYITADKQKN
GIKANFKIRHNVEDGSVQLADHYQQNTPIGDGPVLLPDNH
YLSHQSALSKDPNEKRDHMVLLEFVTAAGITHGMDELYKS
RGPYSIVSPKCGGGGSGPAGILPPKKKRKVSRGRSRLLED
FRNNRYPNLQLREIAGHIMEFSQDQHGSRFIQLKLERATP
AERQLVFNEILQAAYQLMVDVFGNYVIQKFFEFGSLEQKL
ALAERIRGHVLSLALQMYGSRVIEKALEFIPSDQQNEMVR
ELDGHVLKCVKDQNGNHVVQKCIECVQPQSLQFIIDAFKG
QVFALSTHPYGCRVIQRILEHCLPDQTLPILEELHQHTEQ
LVQDQYGSYVIEHVLEHGRPEDKSKIVAEIRGNVLVLSQH
KFANNVVQKCVTHASRTERAVLIDEVCTMNDGPHSALYTM
MKDQYANYVVQKMIDVAEPGQRKIVMHKIRPHIATLRKYT
YGKHILAKLEKYYMKNGVDLG,
nucleotide sequence of PCI-SMN2 reporter
>SEQ ID NO: 8
ATAATTCCCCCACCACCTCCCATATGTCCAGATTCTCTTG
ATGATGCTGATGCTTTGGGAAGTATGTTAATTTCATGGTA
CATGAGTGGCTATCATACTGGCTATTATATGGTAAGTAAT
CACTCAGCATCTTTTCCTGACAATTTTTTTGTAGTTATGT
GACTTTGTTTTGTAAATTTATAAAATACTACTTGCTTCTC
TCTTTATATTACTAAAAAATAAAAATAAAAAAATACAACT
GTCTGAGGCTTAAATTACTCTTGCATTGTCCCTAAGTATA
ATTTTAGTTAATTTTAAAAAGCTTTCATGCTATTGTTAGA
TTATTTTGATTATACACTTTTGAATTGAAATTATACTTTT
TCTAAATAATGTTTTAATCTCTGATTTGAAATTGATTGTA
GGGAATGGAAAAGATGGGATAATTTTTCATAAATGAAAAA
TGAAATTCTTTTTTTTTTTTTTTTTTTTTTGAGACGGAGT
CTTGCTCTGTTGCCCAGGCTGGAGTGCAATGGCGTGATCT
TGGCTCACAGCAAGCTCTGCCTCCTGGATTCACGCCATTC
TCCTGCCTCAGCCTCAGAGGTAGCTGGGACTACAGGTGCC
TGCCACCACGCCTGTCTAATTTTTTGTATTTTTTTGTAAA
GACAGGGTTTCACTGTGTTAGCCAGGATGGTCTCAATCTC
CTGACCCCGTGATCCACCCGCCTCGGCCTTCCAAGAGAAA
TGAAATTTTTTTAATGCACAAAGATCTGGGGTAATGTGTA
CCACATTGAACCTTGGGGAGTATGGCTTCAAACTTGTCAC
TTTATACGTTAGTCTCCTACGGACATGTTCTATTGTATTT
TAGTCAGAACATTTAAAATTATTTTATTTTATTTTATTTT
TTTTTTTTTTTTGAGACGGAGTCTCGCTCTGTCACCCAGG
CTGGAGTACAGTGGCGCAGTCTCGGCTCACTGCAAGCTCC
GCCTCCCGGGTTCACGCCATTCTCCTGCCTCAGCCTCTCC
GAGTAGCTGGGACTACAGGCGCCCGCCACCACGCCCGGCT
AATTTTTTTTTATTTTTAGTAGAGACGGGGTTTCACCGTG
GTCTCGATCTCCTGACCTCGTGATCCACCCGCCTCGGCCT
CCCAAAGTGCTGGGATTACAAGCGTGAGCCACCGCGCCCG
GCCTAAAATTATTTTTAAAAGTAAGCTCTTGTGCCCTGCT
AAAATTATGATGTGATATTGTAGGCACTTGTATTTTTAGT
AAATTAATATAGAAGAAACAACTGACTTAAAGGTGTATGT
TTTTAAATGTATCATCTGTGTGTGCCCCCATTAATATTCT
TATTTAAAAGTTAAGGCCAGACATGGTGGCTTACAACTGT
AATCCCAACAGTTTGTGAGGCCGAGGCAGGCAGATCACTT
GAGGTCAGGAGTTTGAGACCAGCCTGGCCAACATGATGAA
ACCTTGTCTCTACTAAAAATACCAAAAAAAATTTAGCCAG
GCATGGTGGCACATGCCTGTAATCCGAGCTACTTGGGAGG
CTGTGGCAGGAAAATTGCTTTAATCTGGGAGGCAGAGGTT
GCAGTGAGTTGAGATTGTGCCACTGCACTCCACCCTTGGT
GACAGAGTGAGATTCCATCTCAAAAAAAGAAAAAGGCCTG
GCACGGTGGCTCACACCTATAATCCCAGTACTTTGGGAGG
TAGAGGCAGGTGGATCACTTGAGGTTAGGAGTTCAGGACC
AGCCTGGCCAACATGGTGACTACTCCATTTCTACTAAATA
CACAAAACTTAGCCCAGTGGCGGGCAGTTGTAATCCCAGC
TACTTGAGAGGTTGAGGCAGGAGAATCACTTGAACCTGGG
AGGCAGAGGTTGCAGTGAGCCGAGATCACACCGCTGCACT
CTAGCCTGGCCAACAGAGTGAGAATTTGCGGAGGGAAAAA
AAAGTCACGCTTCAGTTGTTGTAGTATAACCTTGGTATAT
TGTATGTATCATGAATTCCTCATTTTAATGACCAAAAAGT
AATAAATCAACAGCTTGTAATTTGTTTTGAGATCAGTTAT
CTGACTGTAACACTGTAGGCTTTTGTGTTTTTTAAATTAT
GAAATATTTGAAAAAAATACATAATGTATATATAAAGTAT
TGGTATAATTTATGTTCTAAATAACTTTCTTGAGAAATAA
TTCACATGGTGTGCAGTTTACCTTTGAAAGTATACAAGTT
GGCTGGGCACAATGGCTCACGCCTGTAATCCCAGCACTTT
GGGAGGCCAGGGCAGGTGGATCACGAGGTCAGGAGATCGA
GACCATCCTGGCTAACATGGTGAAACCCCGTCTCTACTAA
AAGTACAAAAACAAATTAGCCGGGCATGTTGGCGGGCACC
TTTTGTCCCAGCTGCTCGGGAGGCTGAGGCAGGAGAGTGG
CGTGAACCCAGGAGGTGGAGCTTGCAGTGAGCCGAGATTG
TGCCAGTGCACTCCAGCCTGGGCGACAGAGCGAGACTCTG
TCTCAAAAAATAAAATAAAAAAGAAAGTATACAAGTCAGT
GGTTTTGGTTTTCAGTTATGCAACCATCACTACAATTTAA
GAACATTTTCATCACCCCAAAAAGAAACCCTGTTACCTTC
ATTTTCCCCAGCCCTAGGCAGTCAGTACACTTTCTGTCTC
TATGAATTTGTCTATTTTAGATATTATATATAAACGGAAT
TATACGATATGTGGTCTTTTGTGTCTGGCTTCTTTCACTT
AGCATGCTATTTTCAAGATTCATCCATGCTGTAGAATGCA
CCAGTACTGCATTCCTTCTTATTGCTGAATATTCTGTTGT
TTGGTTATATCACATTTTATCCATTCATCAGTTCATGGAC
ATTTAGGTTGTTTTTATTTTTGGGCTATAATGAATAATGT
TGCTATGAACATTCGTTTGTGTTCTTTTTGTTTTTTTGGT
TTTTTGGGTTTTTTTTGTTTTGTTTTTGTTTTTGAGACAG
TCTTGCTCTGTCTCCTAAGCTGGAGTGCAGTGGCATGATC
TTGGCTTACTGCAAGCTCTGCCTCCCGGGTTCACACCATT
CTCCTGCCTCAGCCCGACAAGTAGCTGGGACTACAGGCGT
GTGCCACCATGCACGGCTAATTTTTTGTATTTTTAGTAGA
GATGGGGTTTCACCGTGTTAGCCAGGATGGTCTCGATCTC
CTGACCTCGTGATCTGCCTGCCTAGGCCTCCCAAAGTGCT
GGGATTACAGGCGTGAGCCACTGCACCTGGCCTTAAGTGT
TTTTAATACGTCATTGCCTTAAGCTAACAATTCTTAACCT
TTGTTCTACTGAAGCCACGTGGTTGAGATAGGCTCTGAGT
CTAGCTTTTAACCTCTATCTTTTTGTCTTAGAAATCTAAG
CAGAATGCAAATGACTAAGAATAATGTTGTTGAAATAACA
TAAAATAGGTTATAACTTTGATACTCATTAGTAACAAATC
TTTCAATACATCTTACGGTCTGTTAGGTGTAGATTAGTAA
TGAAGTGGGAAGCCACTGCAAGCTAGTATACATGTAGGGA
AAGATAGAAAGCATTGAAGCCAGAAGAGAGACAGAGGACA
TTTGGGCTAGATCTGACAAGAAAAACAAATGTTTTAGTAT
TAATTTTTGACTTTAAATTTTTTTTTTATTTAGTGAATAC
TGGTGTTTAATGGTCTCATTTTAATAAGTATGACACAGGT
AGTTTAAGGTCATATATTTTATTTGATGAAAATAAGGTAT
AGGCCGGGCACGGTGGCTCACACCTGTAATCCCAGCACTT
TGGGAGGCCGAGGCAGGCGGATCACCTGAGGTCGGGAGTT
AGAGACTAGCCTCAACATGGAGAAACCCCGTCTCTACTAA
AAAAAATACAAAATTAGGCGGGCGTGGTGGTGCATGCCTG
TAATCCCAGCTACTCAGGAGGCTGAGGCAGGAGAATTGCT
TGAACCTGGGAGGTGGAGGTTGCGGTGAGCCGAGATCACC
TCATTGCACTCCAGCCTGGGCAACAAGAGCAAAACTCCAT
CTCAAAAAAAAAAAAATAAGGTATAAGCGGGCTCAGGAAC
ATCATTGGACATACTGAAAGAAGAAAAATCAGCTGGGCGC
AGTGGCTCACGCCGGTAATCCCAACACTTTGGGAGGCCAA
GGCAGGCGAATCACCTGAAGTCGGGAGTTCCAGATCAGCC
TGACCAACATGGAGAAACCCTGTCTCTACTAAAAATACAA
AACTAGCCGGGCATGGTGGCGCATGCCTGTAATCCCAGCT
ACTTGGGAGGCTGAGGCAGGAGAATTGCTTGAACCGAGAA
GGCGGAGGTTGCGGTGAGCCAAGATTGCACCATTGCACTC
CAGCCTGGGCAACAAGAGCGAAACTCCGTCTCAAAAAAAA
AAGGAAGAAAAATATTTTTTTAAATTAATTAGTTTATTTA
TTTTTTAAGATGGAGTTTTGCCCTGTCACCCAGGCTGGGG
TGCAATGGTGCAATCTCGGCTCACTGCAACCTCCGCCTCC
TGGGTTCAAGTGATTCTCCTGCCTCAGCTTCCCGAGTAGC
TGTGATTACAGCCATATGCCACCACGCCCAGCCAGTTTTG
TGTTTTGTTTTGTTTTTTGTTTTTTTTTTTTGAGAGGGTG
TCTTGCTCTGTCCCCCAAGCTGGAGTGCAGCGGCGCGATC
TTGGCTCACTGCAAGCTCTGCCTCCCAGGTTCACACCATT
CTCTTGCCTCAGCCTCCCGAGTAGCTGGGACTACAGGTGC
CCGCCACCACACCCGGCTAATTTTTTTGTGTTTTTAGTAG
AGATGGGGTTTCACTGTGTTAGCCAGGATGGTCTCGATCT
CCTGACCTTTTGATCCACCCGCCTCAGCCTCCCCAAGTGC
TGGGATTATAGGCGTGAGCCACTGTGCCCGGCCTAGTCTT
GTATTTTTAGTAGAGTCGGGATTTCTCCATGTTGGTCAGG
CTGTTCTCCAAATCCGACCTCAGGTGATCCGCCCGCCTTG
GCCTCCAAAAGTGCAAGGCAAGGCATTACAGGCATGAGCC
ACTGTGACCGGCAATGTTTTTAAATTTTTTACATTTAAAT
TTTATTTTTTAGAGACCAGGTCTCACTCTATTGCTCAGGC
TGGAGTGCAAGGGCACATTCACAGCTCACTGCAGCCTTGA
CCTCCAGGGCTCAAGCAGTCCTCTCACCTCAGTTTCCCGA
GTAGCTGGGACTACAGTGATAATGCCACTGCACCTGGCTA
ATTTTTATTTTTATTTATTTATTTTTTTTTGAGACAGAGT
CTTGCTCTGTCACCCAGGCTGGAGTGCAGTGGTGTAAATC
TCAGCTCACTGCAGCCTCCGCCTCCTGGGTTCAAGTGATT
CTCCTGCCTCAACCTCCCAAGTAGCTGGGATTAGAGGTCC
CCACCACCATGCCTGGCTAATTTTTTGTACTTTCAGTAGA
AACGGGGTTTTGCCATGTTGGCCAGGCTGTTCTCGAACTC
CTGAGCTCAGGTGATCCAACTGTCTCGGCCTCCCAAAGTG
CTGGGATTACAGGCGTGAGCCACTGTGCCTAGCCTGAGCC
ACCACGCCGGCCTAATTTTTAAATTTTTTGTAGAGACAGG
GTCTCATTATGTTGCCCAGGGTGGTGTCAAGCTCCAGGTC
TCAAGTGATCCCCCTACCTCCGCCTCCCAAAGTTGTGGGA
TTGTAGGCATGAGCCACTGCAAGAAAACCTTAACTGCAGC
CTAATAATTGTTTTCTTTGGGATAACTTTTAAAGTACATT
AAAAGACTATCAACTTAATTTCTGATCATATTTTGTTGAA
TAAAATAAGTAAAATGTCTTGTGAAACAAAATGCTTTTTA
ACATCCATATAAAGCTATCTATATATAGCTATCTATATCT
ATATAGCTATTTTTTTTAACTTCCTTTATTTTCCTTACAG
GGTTTTAGACAAAATCAAAAAGAAGGAAGGTGCTCACATT
CCTTAAATTAAGGAGTAAGTCTGCCAGCATTATGAAAGTG
AATCTTACTTTTGTAAAACTTTATGGTTTGTGGAAAACAA
ATGTTTTTGAACATTTAAAAAGTTCAGATGTTAGAAAGTT
GAAAGGTTAATGTAAAACAATCAATATTAAAGAATTTTGA
TGCCAAAACTATTAGATAAAAGGTTAATCTACATCCCTAC
TAGAATTCTCATACTTAACTGGTTGGTTGTGTGGAAGAAA
CATACTTTCACAATAAAGAGCTTTAGGATATGATGCCATT
TTATATCACTAGTAGGCAGACCAGCAGACTTTTTTTTATT
GTGATATGGGATAACCTAGGCATACTGCACTGTACACTCT
GACATATGAAGTGCTCTAGTCAAGTTTAACTGGTGTCCAC
AGAGGACATGGTTTAACTGGAATTCGTCAAGCCTCTGGTT
CTAATTTCTCATTTGCAGGAAATGCTGGCATAGAGCAGCA
CTAAATGACACCACTAAAGAAACGATCAGACAGATCTGGA
ATGTGAAGCGTTATAGAAGATAACTGGCCTCATTTCTTCA
AAATATCAAGTGTTGGGAAAGAAAAAAGGAAGTGGAATGG
GTAACTCTTCTTGATTAAAAGTTATGTAATAACCAAATGC
AATGTGAAATATTTTACTGGACTCTATTTTGAAAAACCAT
CTGTAAAAGACTGAGGTGGGGGTGGGAGGCCAGCACGGTG
GTGAGGCAGTTGAGAAAATTTGAATGTGGATTAGATTTTG
AATGATATTGGATAATTATTGGTAATTTTATGAGCTGTGA
GAAGGGTGTTGTAGTTTATAAAAGACTGTCTTAATTTGCA
TACTTAAGCATTTAGGAATGAAGTGTTAGAGTGTCTTAAA
ATGTTTCAAATGGTTTAACAAAATGTATGTGAGGCGTATG
TG,

TABLE 1
RT-PCR Primers

| | SEQ | |
|---|---|---|
| | ID | |
| Name | NO: | Sequence |
| Vector-For | 9 | GCTCTTAAGGCTAGAGTACTTAA |
| | | TACGA |
| SMN2-Inclusion | 10 | CTTCTTTTTGATTTTGTCTAAAACC |
| | | CATATAATAG |
| SMN2-Exclusion | 11 | CTCTATGCCAGCATTTCCATATAA |
| | | TAG |
| GAPDH-Forward | 12 | ACCCAGAAGACTGTGGATGG |
| GAPDH-Reverse | 13 | CAGTGAGCTTCCCGTTCAG |
| ACTB-Forward | 14 | AGATGTGGATCAGCAAGC |
| ACTB-Reverse | 15 | TCATCTTGTTTTCTGCGC |
| SELECT-Forward | 16 | ATGCAGCGACTCAGCCTCTG |
| SELECT-Reverse | 17 | TAGCCAGTACCGTAGTGCGTG |
| ACTB-12- | 18 | tagccagtaccgtagtgcgtg |
| SELECT Probe- | | GAAAGGGTGTAACGCA |
| Up | | ACTAAGTCATAG |
| ACTB-12- | 19 | /5phos/CCGCCTAGAAGCATTT |
| SELECT Probe- | | GCGGcagaggctga |
| Down | | gtcgctgcat |

TABLE 2
gRNAs

| Name | SEQ ID NO: | Sequence |
|---|---|---|
| Non-targeting | 20 | actcaaaaggaagtgacaagaagttgtggaaggtcca |
| gRNA | | gttttgaggggctattacaac |
| On-targeting | 21 | gtaagattcactttcataatgcgttgtggaaggtcca |
| gRNA1 | | gttttgaggggctattacaac |
| On-targeting | 22 | gtagggatgtagattaacctttgttgtggaaggtcca |
| gRNA2 | | gttttgaggggctattacaac |
| On-targeting | 23 | gctggtctgcctactagtgatagttgtggaaggtcca |
| gRNA3 | | gttttgaggggctattacaac |
| On-targeting | 24 | gaagcatttgcggtggacgatggaggggccgttgtgg |
| gRNA5 | | aaggtccagttttgaggggctattacaac |
| On-targeting | 25 | gcctagaagcatttgcggtggagttgtggaaggtcca |
| gRNA6 | | gttttgaggggctattacaac |
| On-targeting | 26 | ctaagtcatagtccgcctagaagttgtggaaggtcca |
| gRNA7 | | gttttgaggggctattacaac |
| On-targeting | 27 | ggtgtaacgcaactaagtcatagttgtggaaggtcca |
| gRNA8 | | gttttgaggggctattacaac |
| On-targeting | 28 | agggtgtaacgcaactaagtcagttgtggaaggtcca |
| gRNA9 | | gttttgaggggctattacaac |
| On-targeting | 29 | aaagggtgtaacgcaactaagtgttgtggaaggtcca |
| gRNA10 | | gttttgaggggctattacaac |
| On-targeting | 30 | agaaagggtgtaacgcaactaagttgtggaaggtcca |
| gRNA11 | | gttttgaggggctattacaac |
| On-targeting | 31 | caagaaagggtgtaacgcaactgttgtggaaggtcca |
| gRNA12 | | gttttgaggggctattacaac |
| On-targeting | 32 | cgcctagaagcatttgcggtgggttgtggaaggtcca |
| gRNA13 | | gttttgaggggctattacaac |
| On-targeting | 33 | cctagaagcatttgcggtggacgttgtggaaggtcca |
| gRNA14 | | gttttgaggggctattacaac |
| On-targeting | 34 | tagaagcatttgcggtggacgagttgtggaaggtcca |
| gRNA15 | | gttttgaggggctattacaac |
| On-targeting | 35 | gaagcatttgcggtggacgatggttgtggaaggtcca |
| gRNA16 | | gttttgaggggctattacaac |
| On-targeting | 36 | agcatttgcggtggacgatggagttgtggaaggtcca |
| gRNA17 | | gttttgaggggctattacaac |
| On-targeting | 37 | gcagccttggttcggtggtcaacgttgtggaaggtcc |
| gRNA18 | | agttttgaggggctattacaac |
| 1xPBSc | 38 | ttgatgta |
| 2xPBSc | 39 | ttgatgtagccttgatgta |
| 3xPBSc-loop | 40 | ttgatgtaAGGGCCCAttgatgtaAGGGCCCAttgat |
| | | gta |
| 5xPBSc | 41 | ttgatgtagccttgatgtagccttgatgtagccttga |
| | | tgtagccttgatgta |
| 5xPBSc-loop | 42 | ttgatgtaAGGGCCCAttgatgtaAGGGCCCAttgat |
| | | gtaAGCGCGCAttgatgtaAGCGCGCAttgatgta |
| 15xPBSc | 43 | ttgatgtagccttgatgtagccttgatgtagccttga |
| | | tgtagccttgatgtaagatttgatgtagccttgatgt |
| | | agccttgatgtagccttgatgtagccttgatgtaaga |
| | | tttgatgtagccttgatgtagccttgatgtagccttg |
| | | atgtagccttgatgta |
| 1xMS2 | 44 | GCGTACACCATCAGGGTACGC |
| 2xMS2 | 45 | GCGTACACCATCAGGGTACGCAGATGCGTACACCATC |
| | | AGGGTACGC |
| 4xMS2 | 46 | GCGTACACCATCAGGGTACGCagatGCGTACACCATC |
| | | AGGGTACGCagatGCGTACACCATCAGGGTACGCaga |
| | | tGCGTACACCATCAGGGTACGC |

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A method of live cell imaging of ribonucleic acid (RNA), comprising:
(a) delivering to a cell an RNA-editing complex that comprises
a catalytically inactive Cas13 (dCas13) nuclease,
a Cas13 guide RNA (gRNA) comprising an RNA aptamer sequence, and
a detectable molecule linked to an RNA-binding domain (RBD) sequence that specifically binds to the RNA aptamer sequence; and
(b) imaging the detectable molecule.

2. The method of claim 1, wherein the dCas13 nuclease is pre-crRNA processing deficient.

3. The method of claim 1 or 2, wherein the dCas13 nuclease is a dCas13b nuclease.

4. The method of any one of the preceding claims, wherein the dCas13 nuclease is a Prevotella dCas13 nuclease.

5. The method of claim 4, wherein the Prevotella dCas13 nuclease is a Prevotella sp. P5-125 dCas13 nuclease (PspdCas13).

6. The method of any one of claims 2-5, wherein the dCas13 nuclease comprises a mutation at one or more position(s) corresponding to amino acid positions 367-370 of the amino acid sequence of SEQ ID NO: 1.

7. The method of claim 6, wherein the mutation at one or more position(s) corresponding to amino acid positions 367-370 of the amino acid sequence of SEQ ID NO: 1 is mutated to a nonpolar neutral amino acid.

8. The method of claim 7, wherein the nonpolar neutral amino acid is alanine.

9. The method of any one of the preceding claims, wherein the RNA aptamer is selected from a Pumilio aptamer sequence, an MS2 aptamer sequence, and a PP7 aptamer sequence.

10. The method of claim 9, wherein the RNA aptamer sequence is a Pumilio aptamer sequence and the RBD sequence is a Pumilio binding domain sequence.

11. The method of claim 9, wherein the RNA aptamer sequence is an MS2 aptamer sequence and the RBD sequence is an MS2 coat protein (MCP) sequence.

12. The method of claim 9, wherein the RNA aptamer sequence is a PP7 aptamer sequence and the RBD sequence is a PP7 coat protein (PCP) sequence.

13. The method of any one of the preceding claims, wherein the Cas13 gRNA binds to a nonrepetitive RNA sequence.

14. A method of targeting ribonucleic acid (RNA) in a live cell, comprising:
(a) delivering to a live cell an RNA-editing complex that comprises
a catalytically inactive Cas13 (dCas13) nuclease,
a Cas13 guide RNA (gRNA) comprising an RNA aptamer sequence, and
an RNA effector molecule linked to an RNA-binding domain (RBD) sequence that specifically binds to the RNA aptamer sequence, optionally wherein the RNA effector molecule is selected from an RNA splicing factor, an RNA methylation or demethylation protein, an RNA degradation molecule, and an RNA processing molecule; and
(b) imaging the detectable molecule.

15. A kit, comprising:
a Cas13 guide RNA (gRNA) linked to an RNA aptamer sequence; and
an RNA effector molecule, optionally a detectable molecule, linked to an RNA-binding domain (RBD) sequence that specifically binds to the RNA aptamer sequence.

16. The kit of claim 15 further comprising a catalytically inactive Cas13 (dCas13) nuclease.

17. A multiplex live cell imaging method, comprising transfecting a cell with:
a first Cas13 guide RNA (gRNA) linked to a first RNA aptamer sequence and a first detectable molecule linked to a first RNA-binding domain (RBD) sequence that specifically binds to the first RNA aptamer sequence; and
a second Cas13 gRNA linked to a second RNA aptamer sequence and an RNA effector molecule, optionally a second detectable molecule, linked to a second RBD sequence that specifically binds to the second RNA aptamer sequence.

18. The method of claim 17 further comprising transfecting the cell with a catalytically inactive Cas13 (dCas13) nuclease.

19. The method of claim 17 or 18, wherein the cell comprises a first RNA of interest and a second RNA of interest, the first Cas13 gRNA specifically binds to the first RNA of interest, and the second Cas13 gRNA specifically binds to the first second of interest.

20. The method of claim 19 further comprising incubating the cell to target, and optionally modify, the first RNA of interest and the second RNA of interest.

21. A composition comprising:
a Cas13 guide RNA (gRNA) comprising a Pumilio binding sequence (PBS), and
a detectable molecule linked to a Pumilio PBS binding domain (PUF domain).

22. A composition comprising:
a first Cas13 guide RNA (gRNA) linked to a first PBS sequence and a first RNA effector molecule, optionally a detectable molecule, linked to a first PUF domain sequence that specifically binds to the first PBS sequence; and
a second Cas13 gRNA linked to a second PBS sequence and a second RNA effector molecule, optionally a detectable molecule, linked to a second PUF domain sequence that specifically binds to the second PBS sequence.

23. The composition of claim 21 or claim 22, further comprising a catalytically inactive Cas13 (dCas13) nuclease.


SEQUENCE LISTING (46 sequences)

SEQ ID NO: 1 - protein, 1090 aa, Artificial Sequence
  Synthetic
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMK

SEQ ID NO: 2 - protein, 1090 aa, Artificial Sequence
  Synthetic
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLAAAATCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMK

SEQ ID NO: 3 - protein, 486 aa, Artificial Sequence
  Synthetic
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRAMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGRGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 4 - protein, 732 aa, Artificial Sequence
  Synthetic
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRAGILPPKKKRKVSRGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGSYVIEHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLGDPKKKRKVDPKKKRKVGGRGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 5 - protein, 787 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRAGILPPKKKRKVSRGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGSYVIEHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLGDPKKKRKVDPKKKRKVGGRGGGGSGGGGSGGGGSGPAQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 6 - protein, 760 aa, Artificial Sequence
  Synthetic
MDYKDHDGDYKDHDIDYKDDDDKIDGGGGSDPKKKRKVDPKKKRKVDPKKKRKVGSTGSRNDGGGGSGGGGSGGGGSGRASRGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGGGRGGGGSGGGGSGGGGSGPAQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 7 - protein, 621 aa, Artificial Sequence
  Synthetic
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSHQSALSKDPNEKRDHMVLLEFVTAAGITHGMDELYKSRGPYSIVSPKCGGGGSGPAGILPPKKKRKVSRGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGSYVIEHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 8 - DNA, 6842 bp, Artificial Sequence
  Synthetic
ataattcccccaccacctcccatatgtccagattctcttgatgatgctgatgctttgggaagtatgttaatttcatggtacatgagtggctatcatactggctattatatggtaagtaatcactcagcatcttttcctgacaatttttttgtagttatgtgactttgttttgtaaatttataaaatactacttgcttctctctttatattactaaaaaataaaaataaaaaaatacaactgtctgaggcttaaattactcttgcattgtccctaagtataattttagttaattttaaaaagctttcatgctattgttagattattttgattatacacttttgaattgaaattatactttttctaaataatgttttaatctctgatttgaaattgattgtagggaatggaaaagatgggataatttttcataaatgaaaaatgaaattcttttttttttttttttttttttgagacggagtcttgctctgttgcccaggctggagtgcaatggcgtgatcttggctcacagcaagctctgcctcctggattcacgccattctcctgcctcagcctcagaggtagctgggactacaggtgcctgccaccacgcctgtctaattttttgtatttttttgtaaagacagggtttcactgtgttagccaggatggtctcaatctcctgaccccgtgatccacccgcctcggccttccaagagaaatgaaatttttttaatgcacaaagatctggggtaatgtgtaccacattgaaccttggggagtatggcttcaaacttgtcactttatacgttagtctcctacggacatgttctattgtattttagtcagaacatttaaaattattttattttattttattttttttttttttttgagacggagtctcgctctgtcacccaggctggagtacagtggcgcagtctcggctcactgcaagctccgcctcccgggttcacgccattctcctgcctcagcctctccgagtagctgggactacaggcgcccgccaccacgcccggctaatttttttttatttttagtagagacggggtttcaccgtggtctcgatctcctgacctcgtgatccacccgcctcggcctcccaaagtgctgggattacaagcgtgagccaccgcgcccggcctaaaattatttttaaaagtaagctcttgtgccctgctaaaattatgatgtgatattgtaggcacttgtatttttagtaaattaatatagaagaaacaactgacttaaaggtgtatgtttttaaatgtatcatctgtgtgtgcccccattaatattcttatttaaaagttaaggccagacatggtggcttacaactgtaatcccaacagtttgtgaggccgaggcaggcagatcacttgaggtcaggagtttgagaccagcctggccaacatgatgaaaccttgtctctactaaaaataccaaaaaaaatttagccaggcatggtggcacatgcctgtaatccgagctacttgggaggctgtggcaggaaaattgctttaatctgggaggcagaggttgcagtgagttgagattgtgccactgcactccacccttggtgacagagtgagattccatctcaaaaaaagaaaaaggcctggcacggtggctcacacctataatcccagtactttgggaggtagaggcaggtggatcacttgaggttaggagttcaggaccagcctggccaacatggtgactactccatttctactaaatacacaaaacttagcccagtggcgggcagttgtaatcccagctacttgagaggttgaggcaggagaatcacttgaacctgggaggcagaggttgcagtgagccgagatcacaccgctgcactctagcctggccaacagagtgagaatttgcggagggaaaaaaaagtcacgcttcagttgttgtagtataaccttggtatattgtatgtatcatgaattcctcattttaatgaccaaaaagtaataaatcaacagcttgtaatttgttttgagatcagttatctgactgtaacactgtaggcttttgtgttttttaaattatgaaatatttgaaaaaaatacataatgtatatataaagtattggtataatttatgttctaaataactttcttgagaaataattcacatggtgtgcagtttacctttgaaagtatacaagttggctgggcacaatggctcacgcctgtaatcccagcactttgggaggccagggcaggtggatcacgaggtcaggagatcgagaccatcctggctaacatggtgaaaccccgtctctactaaaagtacaaaaacaaattagccgggcatgttggcgggcaccttttgtcccagctgctcgggaggctgaggcaggagagtggcgtgaacccaggaggtggagcttgcagtgagccgagattgtgccagtgcactccagcctgggcgacagagcgagactctgtctcaaaaaataaaataaaaaagaaagtatacaagtcagtggttttggttttcagttatgcaaccatcactacaatttaagaacattttcatcaccccaaaaagaaaccctgttaccttcattttccccagccctaggcagtcagtacactttctgtctctatgaatttgtctattttagatattatatataaacggaattatacgatatgtggtcttttgtgtctggcttctttcacttagcatgctattttcaagattcatccatgctgtagaatgcaccagtactgcattccttcttattgctgaatattctgttgtttggttatatcacattttatccattcatcagttcatggacatttaggttgtttttatttttgggctataatgaataatgttgctatgaacattcgtttgtgttctttttgtttttttggttttttgggttttttttgttttgtttttgtttttgagacagtcttgctctgtctcctaagctggagtgcagtggcatgatcttggcttactgcaagctctgcctcccgggttcacaccattctcctgcctcagcccgacaagtagctgggactacaggcgtgtgccaccatgcacggctaattttttgtatttttagtagagatggggtttcaccgtgttagccaggatggtctcgatctcctgacctcgtgatctgcctgcctaggcctcccaaagtgctgggattacaggcgtgagccactgcacctggccttaagtgtttttaatacgtcattgccttaagctaacaattcttaacctttgttctactgaagccacgtggttgagataggctctgagtctagcttttaacctctatctttttgtcttagaaatctaagcagaatgcaaatgactaagaataatgttgttgaaataacataaaataggttataactttgatactcattagtaacaaatctttcaatacatcttacggtctgttaggtgtagattagtaatgaagtgggaagccactgcaagctagtatacatgtagggaaagatagaaagcattgaagccagaagagagacagaggacatttgggctagatctgacaagaaaaacaaatgttttagtattaatttttgactttaaattttttttttatttagtgaatactggtgtttaatggtctcattttaataagtatgacacaggtagtttaaggtcatatattttatttgatgaaaataaggtataggccgggcacggtggctcacacctgtaatcccagcactttgggaggccgaggcaggcggatcacctgaggtcgggagttagagactagcctcaacatggagaaaccccgtctctactaaaaaaaatacaaaattaggcgggcgtggtggtgcatgcctgtaatcccagctactcaggaggctgaggcaggagaattgcttgaacctgggaggtggaggttgcggtgagccgagatcacctcattgcactccagcctgggcaacaagagcaaaactccatctcaaaaaaaaaaaaataaggtataagcgggctcaggaacatcattggacatactgaaagaagaaaaatcagctgggcgcagtggctcacgccggtaatcccaacactttgggaggccaaggcaggcgaatcacctgaagtcgggagttccagatcagcctgaccaacatggagaaaccctgtctctactaaaaatacaaaactagccgggcatggtggcgcatgcctgtaatcccagctacttgggaggctgaggcaggagaattgcttgaaccgagaaggcggaggttgcggtgagccaagattgcaccattgcactccagcctgggcaacaagagcgaaactccgtctcaaaaaaaaaaggaagaaaaatatttttttaaattaattagtttatttattttttaagatggagttttgccctgtcacccaggctggggtgcaatggtgcaatctcggctcactgcaacctccgcctcctgggttcaagtgattctcctgcctcagcttcccgagtagctgtgattacagccatatgccaccacgcccagccagttttgtgttttgttttgttttttgttttttttttttgagagggtgtcttgctctgtcccccaagctggagtgcagcggcgcgatcttggctcactgcaagctctgcctcccaggttcacaccattctcttgcctcagcctcccgagtagctgggactacaggtgcccgccaccacacccggctaatttttttgtgtttttagtagagatggggtttcactgtgttagccaggatggtctcgatctcctgaccttttgatccacccgcctcagcctccccaagtgctgggattataggcgtgagccactgtgcccggcctagtcttgtatttttagtagagtcgggatttctccatgttggtcaggctgttctccaaatccgacctcaggtgatccgcccgccttggcctccaaaagtgcaaggcaaggcattacaggcatgagccactgtgaccggcaatgtttttaaattttttacatttaaattttattttttagagaccaggtctcactctattgctcaggctggagtgcaagggcacattcacagctcactgcagccttgacctccagggctcaagcagtcctctcacctcagtttcccgagtagctgggactacagtgataatgccactgcacctggctaatttttatttttatttatttatttttttttgagacagagtcttgctctgtcacccaggctggagtgcagtggtgtaaatctcagctcactgcagcctccgcctcctgggttcaagtgattctcctgcctcaacctcccaagtagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttacagggttttagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtg

SEQ ID NO: 9 - DNA, 28 bp, Artificial Sequence
  Synthetic
gctcttaaggctagagtacttaatacga

SEQ ID NO: 10 - DNA, 35 bp, Artificial Sequence
  Synthetic
cttctttttgattttgtctaaaacccatataatag

SEQ ID NO: 11 - DNA, 27 bp, Artificial Sequence
  Synthetic
ctctatgccagcatttccatataatag

SEQ ID NO: 12 - DNA, 20 bp, Artificial Sequence
  Synthetic
acccagaagactgtggatgg

SEQ ID NO: 13 - DNA, 19 bp, Artificial Sequence
  Synthetic
cagtgagcttcccgttcag

SEQ ID NO: 14 - DNA, 18 bp, Artificial Sequence
  Synthetic
agatgtggatcagcaagc

SEQ ID NO: 15 - DNA, 18 bp, Artificial Sequence
  Synthetic
tcatcttgttttctgcgc

SEQ ID NO: 16 - DNA, 20 bp, Artificial Sequence
  Synthetic
atgcagcgactcagcctctg

SEQ ID NO: 17 - DNA, 21 bp, Artificial Sequence
  Synthetic
tagccagtaccgtagtgcgtg

SEQ ID NO: 18 - DNA, 49 bp, Artificial Sequence
  Synthetic
tagccagtaccgtagtgcgtggaaagggtgtaacgcaactaagtcatag

SEQ ID NO: 19 - DNA, 40 bp, Artificial Sequence
  Synthetic
ccgcctagaagcatttgcggcagaggctgagtcgctgcat

SEQ ID NO: 20 - DNA, 58 bp, Artificial Sequence
  Synthetic
actcaaaaggaagtgacaagaagttgtggaaggtccagttttgaggggctattacaac

SEQ ID NO: 21 - DNA, 58 bp, Artificial Sequence
  Synthetic
gtaagattcactttcataatgcgttgtggaaggtccagttttgaggggctattacaac

SEQ ID NO: 22 - DNA, 58 bp, Artificial Sequence
  Synthetic
gtagggatgtagattaacctttgttgtggaaggtccagttttgaggggctattacaac

SEQ ID NO: 23 - DNA, 58 bp, Artificial Sequence
  Synthetic
gctggtctgcctactagtgatagttgtggaaggtccagttttgaggggctattacaac

SEQ ID NO: 24 - DNA, 66 bp, Artificial Sequence
  Synthetic
gaagcatttgcggtggacgatggaggggccgttgtggaaggtccagttttgaggggctattacaac

SEQ ID NO: 25 - DNA, 58 bp, Artificial Sequence
  Synthetic
gcctagaagcatttgcggtggagttgtggaaggtccagttttgaggggctattacaac

SEQ ID NO: 26 - DNA, 58 bp, Artificial Sequence
  Synthetic
ctaagtcatagtccgcctagaagttgtggaaggtccagttttgaggggctattacaac

SEQ ID NO: 27 - DNA, 58 bp, Artificial Sequence
  Synthetic
ggtgtaacgcaactaagtcatagttgtggaaggtccagttttgaggggctattacaac

SEQ ID NO: 28 - DNA, 58 bp, Artificial Sequence
  Synthetic
agggtgtaacgcaactaagtcagttgtggaaggtccagttttgaggggctattacaac

SEQ ID NO: 29 - DNA, 58 bp, Artificial Sequence
  Synthetic
aaagggtgtaacgcaactaagtgttgtggaaggtccagttttgaggggctattacaac

SEQ ID NO: 30 - DNA, 58 bp, Artificial Sequence
  Synthetic
agaaagggtgtaacgcaactaagttgtggaaggtccagttttgaggggctattacaac

SEQ ID NO: 31 - DNA, 58 bp, Artificial Sequence
  Synthetic
caagaaagggtgtaacgcaactgttgtggaaggtccagttttgaggggctattacaac

SEQ ID NO: 32 - DNA, 58 bp, Artificial Sequence
  Synthetic
cgcctagaagcatttgcggtgggttgtggaaggtccagttttgaggggctattacaac

SEQ ID NO: 33 - DNA, 58 bp, Artificial Sequence
  Synthetic
cctagaagcatttgcggtggacgttgtggaaggtccagttttgaggggctattacaac

SEQ ID NO: 34 - DNA, 58 bp, Artificial Sequence
  Synthetic
tagaagcatttgcggtggacgagttgtggaaggtccagttttgaggggctattacaac

SEQ ID NO: 35 - DNA, 58 bp, Artificial Sequence
  Synthetic
gaagcatttgcggtggacgatggttgtggaaggtccagttttgaggggctattacaac

SEQ ID NO: 36 - DNA, 58 bp, Artificial Sequence
  Synthetic
agcatttgcggtggacgatggagttgtggaaggtccagttttgaggggctattacaac

SEQ ID NO: 37 - DNA, 59 bp, Artificial Sequence
  Synthetic
gcagccttggttcggtggtcaacgttgtggaaggtccagttttgaggggctattacaac

SEQ ID NO: 38 - DNA, 8 bp, Artificial Sequence
  Synthetic
ttgatgta

SEQ ID NO: 39 - DNA, 19 bp, Artificial Sequence
  Synthetic
ttgatgtagccttgatgta

SEQ ID NO: 40 - DNA, 40 bp, Artificial Sequence
  Synthetic
ttgatgtaagggcccattgatgtaagggcccattgatgta

SEQ ID NO: 41 - DNA, 52 bp, Artificial Sequence
  Synthetic
ttgatgtagccttgatgtagccttgatgtagccttgatgtagccttgatgta

SEQ ID NO: 42 - DNA, 72 bp, Artificial Sequence
  Synthetic
ttgatgtaagggcccattgatgtaagggcccattgatgtaagcgcgcattgatgtaagcgcgcattgatgta

SEQ ID NO: 43 - DNA, 164 bp, Artificial Sequence
  Synthetic
ttgatgtagccttgatgtagccttgatgtagccttgatgtagccttgatgtaagatttgatgtagccttgatgtagccttgatgtagccttgatgtagccttgatgtaagatttgatgtagccttgatgtagccttgatgtagccttgatgtagccttgatgta

SEQ ID NO: 44 - DNA, 21 bp, Artificial Sequence
  Synthetic
gcgtacaccatcagggtacgc

SEQ ID NO: 45 - DNA, 46 bp, Artificial Sequence
  Synthetic
gcgtacaccatcagggtacgcagatgcgtacaccatcagggtacgc

SEQ ID NO: 46 - DNA, 96 bp, Artificial Sequence
  Synthetic
gcgtacaccatcagggtacgcagatgcgtacaccatcagggtacgcagatgcgtacaccatcagggtacgcagatgcgtacaccatcagggtacgc